(19) United States
(12) Patent Application Publication
(10) Pub. No.: US 2023/0233586 A1
(43) Pub. Date: Jul. 27, 2023

(54) COMPOUNDS AND METHODS FOR TREATING FUNGAL INFECTIONS

(71) Applicant: Amplyx Pharmaceuticals, Inc., New York, NY (US)

(72) Inventors: Karen Joy Shaw, Poway, CA (US); Haran Schlamm, Rancho Santa Fe, CA (US); Michael R. Hodges, San Diego, CA (US)

(73) Assignee: Amplyx Pharmaceuticals, Inc., New York, NY (US)

(21) Appl. No.: 18/002,012

(22) PCT Filed: Jun. 16, 2021
(86) PCT No.: PCT/US2021/037578

§ 371 Date: Dec. 15, 2022

Related U.S. Application Data
(60) Provisional application No. 63/040,450, filed on Jun. 17, 2020.

(51) Int. Cl. A61K 31/675; A61K 31/444; A61P 31/10; A61K 9/00

(52) U.S. Cl. CPC A61K 31/675; A61K 31/444; A61P 31/10; A61K 9/0019; A61K 9/0053

(57) ABSTRACT

Provided herein are compositions and methods of use thereof for the treatment of fungal infections and diseases.

### CROSS-REFERENCE TO RELATED APPLICATIONS

This application claims the benefit of U.S. Provisional Application No. 63/040,450, filed Jun. 17, 2020, which is incorporated herein by reference in its entirety.

### FIELD OF THE INVENTION

Fungi infect humans and are a major cause of human health problems. The present disclosure generally relates to the treatment of fungal infections in humans.

### BACKGROUND

Fungi infect humans and are a major cause of human health problems. They also infect plants and cause enormous losses in agricultural productivity. The present disclosure generally relates to the treatment and/or prevention of fungal infections and diseases.

### SUMMARY OF THE INVENTION

In one aspect, described herein is a method of treating a fungal infection in a subject, the method comprising administering to a subject with a fungal infection a therapeutically effective amount of compound 1:

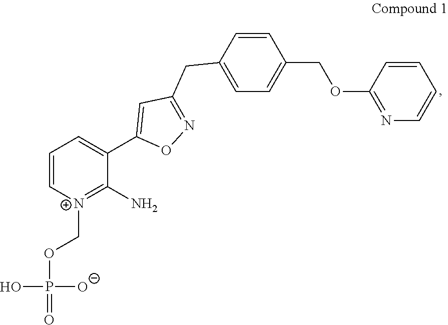 [SMILES: Nc1c(-c2cc(Cc3ccc(COc4ccccn4)cc3)no2)ccc[n+]1COP(=O)([O-])O]

or a pharmaceutically acceptable salt, solvate, or hydrate thereof; wherein the subject has a contradiction to standard of care antifungal therapy. In some embodiments, the contradiction to standard of care antifungal therapy is due to compromised renal function. Standard of care therapies (amphotericin B and voriconazole) can cause renal toxicity.

In another aspect, described herein is a method of treating a fungal infection in a subject, the method comprising administering to a subject with a fungal infection a therapeutically effective amount of Compound 1, or a pharmaceutically acceptable salt, solvate, or hydrate thereof; wherein a dose adjustment of the compound 1, or a pharmaceutically acceptable salt, solvate, or hydrate thereof that is administered to the subject is not required based on the kidney status of the subject. In some embodiments, the fungal infection is an invasive fungal infection. In some embodiments, the fungal infection is candidiasis. In some embodiments, the fungal infection is aspergillosis.

In another aspect, described herein is a method of treating a fungal infection in a subject, the method comprising administering to a subject with a fungal infection a therapeutically effective amount of compound 1, or a pharmaceutically acceptable salt, solvate, or hydrate thereof; wherein the subject has kidney disease. In some embodiments, kidney disease comprises renal impairment. In some embodiments, described herein is a method of treating a fungal infection in a subject, the method comprising administering to a subject with a fungal infection a therapeutically effective amount of compound 1, or a pharmaceutically acceptable salt, solvate, or hydrate thereof; wherein the subject has renal impairment and no dose adjustment of compound 1, or a pharmaceutically acceptable salt, solvate, or hydrate thereof, is required. In some embodiments, no dose adjustment is needed in subjects with mild, moderate, or severe renal impairment. In some embodiments, the therapeutically effective amount of compound 1, or a pharmaceutically acceptable salt, solvate, or hydrate thereof, provides a steady state 24-hr Area Under the Concentration-Time Curve (AUC₀₋₂₄) of compound 1A in the subject of at least about 100 μg×hr/mL, at least about 150 μg×hr/mL, at least about 200 μg×hr/mL, or at least about 250 μg×hr/mL:

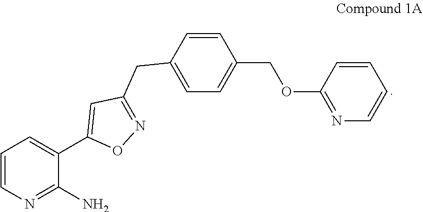 [SMILES: Nc1ncccc1-c1cc(Cc2ccc(COc3ccccn3)cc2)no1]

In some embodiments, the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks. In some embodiments, the treatment regimen comprises the administration of a loading dose followed by daily maintenance doses. In some embodiments, a loading dose of compound 1, or a pharmaceutically acceptable salt, solvate, or hydrate thereof, comprises at least about 2000 mg/day of Compound 1. In some embodiments, maintenance doses comprise at least about 600 mg/day, at least about 700 mg/day, at least about 800 mg/day, at least about 900 mg/day, or at least about 1000 mg/day of Compound 1.

In one aspect, described herein is a method of treating a fungal infection in a subject, the method comprising administering to a subject with a fungal infection a therapeutically effective amount of compound 1, or a pharmaceutically acceptable salt, solvate, or hydrate thereof; wherein, the fungal infection in the subject is caused by Candida spp., Aspergillus spp., Scedosporium spp., Fusarium spp., Paecilomyces spp., Purpureocillium spp., Dematiaceous spp., Rhizopus, Mucor spp., Lichtheimia spp., Cunninghamella spp., Acremonium spp., Rasamsonia spp., Scedosporium spp., Schizophyllum spp., Trichoderma spp., Alternaria spp., Cladophialophora spp., Cladosporium spp., Exophiala spp., Fonsecaea spp., Lomentospora spp., Phialophora spp., Scopulariopsis spp., Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., Wickerhamomyces (Pichia) spp., Emergomyces spp., Talaromyces spp., or Emmonsia-like fungi, or a combination thereof; the therapeutically effective amount of compound 1 provides a steady state 24-hr Area Under the Concentration-Time Curve (AUC₀₋₂₄) of compound 1A in the subject of at least about 150 μg×hr/mL of compound 1A; wherein the subject has a contradiction to standard of care antifungal therapy; and wherein the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks.

In another aspect, described herein is a method of treating a fungal infection in a subject, the method comprising administering to a subject with a fungal infection a therapeutically effective amount of compound 1, or a pharmaceutically acceptable salt, solvate, or hydrate thereof; wherein, the fungal infection in the subject is caused by Candida spp., Aspergillus spp., Scedosporium spp., Fusarium spp., Paecilomyces spp., Purpureocillium spp., Dematiaceous spp., Rhizopus, Mucor spp., Lichtheimia spp., Cunninghamella spp., Acremonium spp., Rasamsonia spp., Scedosporium spp., Schizophyllum spp., Trichoderma spp., Alternaria spp., Cladophialophora spp., Cladosporium spp., Exophiala spp., Fonsecaea spp., Lomentospora spp., Phialophora spp., Scopulariopsis spp., Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., Wickerhamomyces (Pichia) spp., Emergomyces spp., Talaromyces spp., or Emmonsia-like fungi, or a combination thereof; the therapeutically effective amount of compound 1 provides a steady state 24-hr Area Under the Concentration-Time Curve (AUC₀₋₂₄) of compound 1A in the subject of at least about 100 μg×hr/mL of compound 1, wherein the subject has a contradiction to standard of care antifungal therapy; and wherein the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks.

In another aspect, described herein is a method of treating a fungal infection in a subject, the method comprising administering to a subject with a fungal infection a therapeutically effective amount of compound 1, or a pharmaceutically acceptable salt, solvate, or hydrate thereof; wherein, the fungal infection in the subject is caused by Candida spp., Aspergillus spp., Scedosporium spp., Fusarium spp., Paecilomyces spp., Purpureocillium spp., Dematiaceous spp., Rhizopus, Mucor spp., Lichtheimia spp., Cunninghamella spp., Acremonium spp., Rasamsonia spp., Scedosporium spp., Schizophyllum spp., Trichoderma spp., Alternaria spp., Cladophialophora spp., Cladosporium spp., Exophiala spp., Fonsecaea spp., Lomentospora spp., Phialophora spp., Scopulariopsis spp., Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., Wickerhamomyces (Pichia) spp., Emergomyces spp., Talaromyces spp., or Emmonsia-like fungi, or a combination thereof; wherein the subject has a contradiction to standard of care antifungal therapy; and wherein the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks.

In some embodiments, the contradiction to standard of care antifungal therapy is due to reduced kidney function.

In some embodiments, the contradiction to standard of care antifungal therapy is due to a kidney disease in the subject.

In some embodiments, the kidney disease is chronic kidney disease, metabolic syndrome, vesicoureteral reflux, tubulointerstitial renal fibrosis, IgA nephropathy, diabetic nephropathy, Alport syndrome, HIV associated nephropathy, glomerular nephritis (GN), focal segmental glomerulosclerosis, membranous glomerulonephritis, mesangiocapillary GN, interstitial fibrosis and tubular atrophy (IFTA), acute kidney injury (AKI), acute obstructive nephropathy, or drug induced fibrosis.

In some embodiments, the kidney disease is chronic kidney disease (CKD). In some embodiments, the chronic kidney disease (CKD) is Stage 1 CKD, Stage 2 CKD, Stage 3 CKD, Stage 4 CKD, or Stage 5 CKD.

In some embodiments, the subject has high levels of protein in his or her urine (proteinuria).

In some embodiments, the therapeutically effective amount of compound 1 provides a steady state 24-hr Area Under the Concentration-Time Curve (AUC₀₋₂₄) of at least 50 μg×hr/mL of the compound 1A. In some embodiments, the therapeutically effective amount of compound 1 provides a steady state 24-hr Area Under the Concentration-Time Curve (AUC₀₋₂₄) of at least 100 μg×hr/mL of the compound 1A. In some embodiments, the therapeutically effective amount of compound 1 provides a steady state 24-hr Area Under the Concentration-Time Curve (AUC₀₋₂₄) of at least 150 g×hr/mL of the compound 1A. In some embodiments, the therapeutically effective amount of compound 1 provides a steady state 24-hr Area Under the Concentration-Time Curve (AUC₀₋₂₄) of at least 200 g×hr/mL of the compound 1A.

In some embodiments, the contradiction to standard of care antifungal therapy comprises an azole antifungal, an allylamine antifungal agent, echinocandin antifungal, or polyene antifungal.

In some embodiments, the contradiction to standard of care antifungal therapy comprises amphotericin B, candicidin, filipin, hamycin, natamycin, nystatin, rimocidin, bifonazole, butoconazole, clotrimazole, econazole, fenticonazole, isavuconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, or terbinafine, anidulafungin, caspofungin, micafungin, rezafungin, or a pharmaceutically acceptable salt of any of the preceding antifungal agents.

In some embodiments, the fungal infection is caused by Candida spp., Aspergillus spp., Scedosporium spp., Fusarium spp., Paecilomyces spp., Purpureocillium spp., Dematiaceous spp., or Mucorales fungi, or a combination thereof.

In some embodiments, the subject is immunocompromised.

In some embodiments, the subject is infected with HIV/AIDS or has cancer.

In some embodiments, the cancer is acute myeloid leukemia or acute lymphoid leukemia.

In some embodiments, the subject has neutropenia.

In some embodiments, the subject has lymphopenia.

In some embodiments, the subject is undergoing or has undergone cancer chemotherapy treatment.

In some embodiments, the subject is undergoing or has undergone corticosteroid treatment.

In some embodiments, the subject is undergoing or has undergone TNF inhibitor treatment.

In some embodiments, the subject is an organ transplant recipient.

In some embodiments, the subject is a hematopoietic stem-cell transplant recipient.

In some embodiments, the subject has graft-versus-host disease.

In some embodiments, the fungal infection is superficial, locally invasive, or disseminated throughout the subject.

In some embodiments, the fungal infection is a cutaneous infection, lung infection, sinus infection, central nervous system infection, brain infection, eye infection, heart infection, kidney infection, gastrointestinal tract infection, stomach infection, pelvic infection, blood infection, or a combination thereof.

In some embodiments, the fungal infection comprises a fungal disease or condition that is candidiasis, aspergillosis, blastomycosis, coccidioidomycosis (Valley Fever), cryptococcosis, histoplasmosis, mucormycosis, Pneumocystis pneumonia (PCP), ringworm, sporotrichosis, talaromycosis, allergic bronchopulmonary aspergillosis, allergic sinusitis, azole-resistant A. fumigatus, aspergilloma, pulmonary aspergillosis, invasive aspergillosis, cutaneous aspergillosis, fusariosis, scedosporiosis, rhinocerebral mucormycosis, pulmonary mucormycosis, disseminated mucormycosis, abdominal-pelvic mucormycosis, gastric mucormycosis, cutaneous mucormycosis, or a combination thereof.

In some embodiments, the treatment regimen comprises a loading dose of compound 1, or a pharmaceutically acceptable salt, solvate, or hydrate thereof, and a maintenance dose of compound 1, or a pharmaceutically acceptable salt, solvate, or hydrate thereof.

In some embodiments, the treatment regimen comprises a loading dose of compound 1, or a pharmaceutically acceptable salt, solvate, or hydrate thereof, of about 2000 mg compound 1, or a pharmaceutically acceptable salt, solvate, or hydrate thereof.

In some embodiments, the loading dose of compound 1, or a pharmaceutically acceptable salt, solvate, or hydrate thereof, is administered to the subject by intravenous (I.V.) infusion.

In some embodiments, the loading dose of compound 1, or a pharmaceutically acceptable salt, solvate, or hydrate thereof, comprises the administration of two doses of compound 1, or a pharmaceutically acceptable salt, solvate, or hydrate thereof, to the subject by intravenous (I.V.) infusion.

In some embodiments, each loading dose of compound 1, or a pharmaceutically acceptable salt, solvate, or hydrate thereof, is administered to the subject by intravenous (I.V.) infusion over about 30 minutes to about 4 hours.

In some embodiments, each dose of the loading dose comprises about 1000 mg of compound 1, or a pharmaceutically acceptable salt, solvate, or hydrate thereof.

In some embodiments, the loading dose comprises administration of about 1000 mg of compound 1, or a pharmaceutically acceptable salt, solvate, or hydrate thereof, to the subject by intravenous (I.V.) infusion followed by a second administration of about 1000 mg of compound 1, or a pharmaceutically acceptable salt, solvate, or hydrate thereof, to the subject by intravenous (I.V.) infusion within about 24 hours of the first infusion.

In some embodiments, the maintenance dose is administered once daily starting on the second day of treatment.

In some embodiments, the maintenance dose comprises once daily administration of about 600 mg to about 1500 mg of compound 1, or a pharmaceutically acceptable salt, solvate, or hydrate thereof.

In some embodiments, the maintenance dose of compound 1, or a pharmaceutically acceptable salt, solvate, or hydrate thereof is administered over a period of about 30 minutes to about 4 hours by I.V. infusion starting on the second, third, or fourth day of treatment.

In some embodiments, the maintenance dose of about 600 mg to about 1200 mg compound 1, or a pharmaceutically acceptable salt, solvate, or hydrate thereof is administered over a period of about 30 minutes to about 4 hours by I.V. infusion starting on the second, third, or fourth day of treatment.

In some embodiments, the maintenance dose of compound 1, or a pharmaceutically acceptable salt, solvate, or hydrate thereof is administered orally to the subject starting on the second, third, or fourth day of treatment.

In some embodiments, the maintenance dose of about 800 mg to about 1000 mg compound 1, or a pharmaceutically acceptable salt, solvate, or hydrate thereof is administered orally once daily to the subject starting on the second, third, or fourth day of treatment.

In some embodiments, starting on the second, third, or fourth day of treatment: a) about 600 mg to about 900 mg of compound 1, or a pharmaceutically acceptable salt, solvate, or hydrate thereof is administered over a period of about 30 minutes to about 3 hours by I.V. infusion; or b) about 700 mg to about 1000 mg of compound 1, or a pharmaceutically acceptable salt, solvate, or hydrate thereof is administered orally once daily.

In some embodiments, starting on the second day of treatment, about 600 mg to about 900 mg of compound 1, or a pharmaceutically acceptable salt, solvate, or hydrate thereof is administered over a period of about 30 minutes to about 3 hours by I.V. infusion; and starting on the fourth day of treatment: a) about 600 mg to about 900 mg of compound 1, or a pharmaceutically acceptable salt, solvate, or hydrate thereof is administered over a period of about 30 minutes to about 3 hours by I.V. infusion; or b) about 700 mg to about 1000 mg of compound 1, or a pharmaceutically acceptable salt, solvate, or hydrate thereof is administered orally once daily.

In some embodiments, the compound 1, or a pharmaceutically acceptable salt, solvate, or hydrate thereof is administered in combination with an additional therapeutic agent.

In some embodiments, the treatment regimen comprises the daily administration of compound 1, or a pharmaceutically acceptable salt, solvate, or hydrate thereof, for about 4 weeks to about 6 weeks.

In some embodiments, the treatment regimen comprises the daily administration of compound 1, or a pharmaceutically acceptable salt, solvate, or hydrate thereof, for about 4 weeks to about 12 weeks.

In some embodiments, the treatment regimen comprises a loading dose of compound 1, or a pharmaceutically acceptable salt, solvate, or hydrate thereof, and maintenance doses of compound 1, or a pharmaceutically acceptable salt, solvate, or hydrate thereof; wherein the loading dose of compound 1, or a pharmaceutically acceptable salt, solvate, or hydrate thereof, comprises the administration of two doses of compound 1, or a pharmaceutically acceptable salt, solvate, or hydrate thereof, to the subject by intravenous (I.V.) infusion on the first day of therapy, wherein each dose comprises about 1000 mg of compound 1, or a pharmaceutically acceptable salt, solvate, or hydrate thereof; followed by maintenance doses comprising once daily administration of about 600 mg of compound 1, or a pharmaceutically acceptable salt, solvate, or hydrate thereof by intravenous (I.V.) infusion for at least two days, followed by either: once daily administration of about 600 mg of compound 1, or a pharmaceutically acceptable salt, solvate, or hydrate thereof, by intravenous (I.V.) infusion; or once daily oral administration of about 700 mg of compound 1, or a pharmaceutically acceptable salt, solvate, or hydrate thereof.

In some embodiments, the treatment regimen comprises up to 14 days of administration of compound 1, or a pharmaceutically acceptable salt, solvate, or hydrate thereof.

In some embodiments, the fungal infection in the subject is caused by Candida spp.

In some embodiments, the fungal infection in the subject is caused by Candida spp. and the treatment regimen comprises up to 14 days of administration of compound 1, or a pharmaceutically acceptable salt, solvate, or hydrate thereof.

In some embodiments, the treatment regimen increases the chances of survival for the subject, decreases galactomannan levels in the subject, decreases β-d-glucan levels in the subject, or a combination thereof.

In some embodiments, a dose adjustment of the compound 1, or a pharmaceutically acceptable salt, solvate, or hydrate thereof that is administered to the subject is not required based on the kidney status of the subject.

Articles of manufacture, which include packaging material, compound 1, or a pharmaceutically acceptable salt, solvate, or hydrate thereof, within the packaging material, and a label that indicates that compound 1, or a pharmaceutically acceptable salt, solvate, or hydrate thereof, is used for treating a fungal infection, or for the prevention or amelioration of one or more symptoms of a fungal infection are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION

Provided herein are, for example, compositions for treating and/or preventing a fungal infection or disease. Also provided herein are, for example, methods of treating and/or preventing a fungal infection or disease.

Many patients have limited or no antifungal treatment options due to documented/anticipated resistance, contraindication, intolerance, or lack of clinical response to standard of care (SOC) antifungal therapy. Under this setting, compound 1 (shown below) has advantages over SOC antifungal therapy and thus supports its preliminary investigation for the treatment of invasive mold infections (IMIs).

Compound 1, a prodrug rapidly converted in vivo by phosphatases to the microbiologically active moiety compound 1A, is a broad-spectrum antifungal agent in for the treatment of invasive fungal infections by both intravenous and oral routes of administration. Compound 1 is a pro-drug with a labile phosphate moiety. The phosphate moiety improves the aqueous solubility of the drug substance at a higher pH range, but also has limited stability.

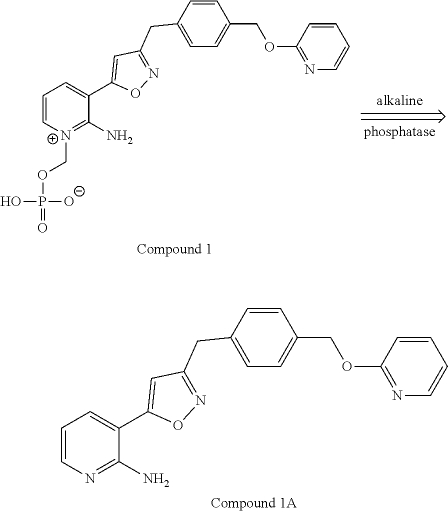 [SMILES: Nc1c(-c2cc(Cc3ccc(COc4ccccn4)cc3)no2)ccc[n+]1COP(=O)([O-])O ; Nc1ncccc1-c1cc(Cc2ccc(COc3ccccn3)cc2)no1]

Compound 1A inhibits the fungal glycosylphosphatidylinositol (GPI)-anchored wall transfer protein 1 (GWT1) enzyme, a highly conserved inositol acylase that catalyzes an early step in the GPI-anchored biosynthesis pathway. This inhibition has pleiotropic effects on the fungal cell due to inhibition of cell wall mannoprotein localization, which comprises cell wall integrity, biofilm formation, germ tube formation, and fungal growth. Compound 1A does not inhibit phosphatidylinositol glycan anchor biosynthesis class W (PIGW) protein, the closest mammalian ortholog of the fungal GWT1 protein consistent with the potential for a significant target-based therapeutic window.

Compound 1A has demonstrated broad in vitro antifungal activity against Candida spp., Cryptococcus spp., Aspergillus spp., Scedosporium spp., Fusarium spp., and some Mucorales fungi, including activity against azole- and echinocandin-resistant strains. In 5-fluorouracil immunosuppressed mice with IMIs (Aspergillus fumigatus, Scedosporium prolificans, and Fusarium solani), compound 1 or 1A demonstrated statistically significantly improved survival rates and reduced pulmonary fungal colony counts. In cyclophosphamide and cortisone acetate immunosuppressed mice with IMIs (A. fumigatus, S. apiospermum, F. solani, and Rhizopus spp.), compound 1 demonstrated statistically significantly improved survival rates and reduced fungal burden.

Additionally, Compound 1A has demonstrated antifungal activity against a broad range of clinical isolates of rare mold infections and rare yeast infections, including activity against a range of Aspergillus spp., Scedosporium spp., Fusarium spp., Paecilomyces spp., Purpureocillium spp., Dematiaceous spp., Mucorales fungi, Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., Wickerhamomyces (Pichia) spp., Emergomyces spp., Talaromyces spp., or Emmonsia-like fungi. These rare mold and rare yeast species generally pose no threat to healthy subjects, but can lead to invasive mold infections in immunocompromised individuals.

In some embodiments, Compound 1 or Compound 1A is used in the treatment of a variety of fungal infections caused by Candida, Cryptococcus, Blastomyces, Histoplasma, Coccidioides, or a combination thereof.

In some embodiments, Compound 1 or Compound 1A is used in the treatment of a variety of mold and rare mold infections. In some embodiments, the mold or rare mold is caused by Aspergillus spp., Mucorales fungi, Hyalohyphomycete fungi, Phaeohyphomycete fungi, or a combination thereof.

Aspergillus spp. include A. flavus, A niger, A. fumigatus, A. terreus.

Mucorales fungi include Rhizopus spp., Mucor spp., Lichtheimia spp., Cunninghamella spp.

Hyalohyphomycete fungi include Acremonium spp., Fusarium spp., Paecilomyces spp., Rasamsonia, spp., Scedosporium spp., Schizophyllum spp., Trichoderma spp.

Phaeohyphomycete fungi include Alternaria spp., Cladophialophora spp., Cladosporium spp., Exophiala spp., Fonsecaea spp., Lomentospora spp., Phialophora spp., Scopulariopsis spp.

Scedosporium spp. include S. apiospermum, S. boydii, S. dehoogii.

Fusarium spp. include F. solani.

Rhizopus spp. include Rhizopus oryzae.

In some embodiments, Compound 1 or Compound 1A is used in the treatment of infections caused by Aspergillus spp., Scedosporium spp., Fusarium spp., Paecilomyces spp., Purpureocillium spp., Dematiaceous spp., or Mucorales fungi, or a combination thereof; including A. flavus, A niger, A. fumigatus, A. terreus, S. apiospermum, S. boydii, S. dehoogii, F. solani, P. lilacinus, P. variotii, and Rhizopus oryzae.

In some embodiments, Compound 1 or Compound 1A is used in the treatment of a variety of yeast and rare yeast infections, including those caused by Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., or Wickerhamomyces (Pichia) spp., or a combination thereof, including G. clavatum, T. asahii, T. mucoides, T. mycotoxinivorans, M. furfur, R. mucilaginosa, or S. cerevisiae.

In some embodiments, Compound 1 or Compound 1A is used in the treatment of a variety of additional fungal infections, including dimorphic fungal infections caused by Emergomyces spp., Talaromyces spp., or Emmonsia-like fungi, or a combination thereof, including T. marneffei.

Pharmacokinetic-pharmacodynamic (PK-PD) studies in immunosuppressed mice with invasive infections caused by A. fumigatus have shown that the area under the concentration-time curve (AUC) divided by the minimal effective concentration (MEC) ratio is the driver of efficacy. The dose regimen employed in this study provides a steady state AUC≥200 μg×hr/mL of compound 1, or active metabolite of compound 1 (i.e., compound 1A), which is associated with efficacy (colony count and survival benefit) in immunocompromised mice with invasive pulmonary aspergillosis (IPA). Additionally, formal PK-PD studies demonstrated that the dose regimen has favorable probability of target attainment (PTA) for the majority of isolates anticipated to be encountered in this study.

In Phase 1 clinical studies of compound 1, the safety, tolerability, and PK of single and multiple ascending doses administered intravenously (IV) and orally (PO) have been studied. To date, a total of 197 healthy volunteers and 21 patients with acute myeloid leukemia (AML) have received compound 1 across 5 Phase 1 studies. The duration of the multiple-dose regimens in these studies was 7, 14, and 42 days (6 weeks).

Compound 1 may have potential benefits compared to the current SOC for treatment of invasive infections caused by Candida spp., including candidemia and Aspergillus spp. or rare molds. Furthermore, compound 1 has a differentiated safety profile, is available as IV and PO formulation, and may have fewer DDIs than the SOC treatments.

Patients with azole-resistant mold infections, including azole-resistant Candida spp., A. fumigatus and some rare molds (e.g., Fusarium spp., Scedosporium spp., species of the Mucorales order) typically receive IV treatment with a polyene. Polyenes have been associated with risk of nephrotoxicity, electrolyte imbalance, and infusion reactions which can be limiting in patient care. Compound 1 has broad-spectrum antifungal activity with coverage against azole-resistant molds, and has the potential to be safer and easier to use compared to polyene.

In some embodiments, compound 1 provides an advantage over a polyene for the treatment of “breakthrough” infections in patients receiving prophylaxis with mold active triazoles. Compound 1 has the potential to provide antifungal coverage for Candida spp, A. fumigatus and rare molds, without the potential for polyene-induced toxicities. With wide tissue penetration, compound 1 may provide a benefit for the treatment of patients with invasive fungal infections in the eye and central nervous system.

In some embodiments, compound 1 provides a benefit to patients with invasive fungal infections who are unable to receive treatment with a mold-active azole due to intolerance, toxicity, or clinically significant drug interactions. Compound 1 has the potential to provide broad-spectrum antifungal coverage, without the risk of hepatic or other azole-associated toxicities, and is expected to be less likely to induce clinically significant drug interactions.

Compound 1 has a novel mechanism of action with broad spectrum activity against Candida spp. (yeast) and Aspergillus spp. (mold), including activity against polyene and azole-resistant strains of Aspergillus spp. Compound 1 has demonstrated efficacy in a number of animal models of IMIs, including Aspergillus spp., Fusarium spp., Scedosporium spp., and species from the Mucorales order. Compound 1 is available in both IV and PO formulations with wide-tissue distribution, including the eye and central nervous system, and has been safe and well tolerated with a favorable safety and drug-drug interaction (DDI) profile that is differentiated from SOC antifungal therapy. Compound 1 has the potential to be used as a first-line agent for the treatment of IMIs through a unique mechanism of action. Thus, compound 1 has potential to fill an unmet need for patients with limited or no antifungal treatment options due to documented/anticipated resistance, contraindication, intolerance, or lack of clinical response to standard of care (SOC) antifungal therapy.

Nephrotoxicity is one of the more problematic adverse effects of antifungal therapy. Drug-induced kidney injury is among the reasons for compound attrition in drug development. It is a common adverse effect of amphotericin B, which is regarded as the “gold standard” antifungal agent (Kuznar W., Baglin T. (2015). MD Conf. Express 13 (13), 12-13). Therefore, the nephrotoxic effect of existing antifungal agents, particularly of amphotericin B, has been extensively studied using in vitro and in vivo models (van Etten et al. J Antimicrob Chemother. 1993 November; 32(5):723-39).

Patients with significant kidney dysfunction on amphotericin B therapy will require persistent dialysis after discontinuation of the antifungal (Groll et al. Adv Pharmacol 1998; 44:343-500). A patient's risk of developing severe kidney damage during amphotericin B therapy depends on the dose and duration of amphotericin B, underlying health and fluid status of the patient, previous or underlying kidney disease, and the receipt of other potentially nephrotoxic drugs (e.g., aminoglycoside antibiotics, radiocontrast dye, cyclosporine, etc.).

In some cases, drug-induced kidney injury resulting from antifungal therapy, especially among those patients with invasive fungal infections, results in an increased risk of death and prolonged hospital stay.

In some embodiments, standard of care (SOC) antifungal therapy is contraindicated in patents with kidney diseases and/or with underlying medical conditions that lead to kidney dysfuntion such as renal insufficiency or impairment. Examples of such diseases and insults include chronic kidney disease, metabolic syndrome, vesicoureteral reflux, tubulointerstitial renal fibrosis, IgA nephropathy, diabetes (including diabetic nephropathy), Alport syndrome, HIV associated nephropathy, resultant glomerular nephritis (GN), including, but not limited to, focal segmental glomerulosclerosis and membranous glomerulonephritis, mesangiocapillary GN and resultant interstitial fibrosis and tubular atrophy (IFTA), including but not limited to, recovery post acute kidney injury (AKI), acute obstructive nephropathy and drug induced fibrosis, and resultant glomerular nephritis (GN), including, but not limited to, focal segmental glomerulosclerosis and membranous glomerulonephritis.

Glomerulonephritis, which causes inflammation in glomeruli, is a common cause of end-stage renal failure. Severe and prolonged inflammation can damage glomeruli and lead to kidney damage. Connective tissue growth factor (CTGF) is a member of the CCN matricellular protein family, consisting of four domains, that regulates the signaling of other growth factors and promotes kidney damage.

It has become recognized that metabolic syndrome is a cluster of abnormalities including diabetic hallmarks such as insulin resistance, as well as central or visceral obesity and hypertension. In nearly all cases, dysregulation of glucose results in the stimulation of cytokine release and upregulation of extracellular matrix deposition. Additional factors contributing to chronic kidney disease, diabetes, metabolic syndrome, and glomerular nephritis include hyperlipidemia, hypertension, and proteinuria, all of which result in further damage to the kidneys and further stimulate the extracellular matrix deposition. Thus, regardless of the primary cause, insults to the kidneys may result in kidney fibrosis and the concomitant loss of kidney function. (Schena, F. and Gesualdo, L., Pathogenic Mechanisms of Diabetic Nephropathy, J. Am. Soc. Nephrol., 16: S30-33 (2005); Whaley-Connell, A., and Sower, J. R., Chronic Kidney Disease and the Cardiometabolic Syndrome, J. Clin. Hypert., 8(8): 546-48 (2006)).

In some embodiments, therapy with Compound 1 is not contraindicated in subjects with already compromised kidney function. In some embodiments, therapy with Compound 1 is not contraindicated in subjects with kidney disease. In some embodiments, the kidney disease is chronic kidney disease (CKD). In some embodiments, the kidney disease is Alport syndrome.

In some embodiments, therapy with Compound 1 is not contraindicated in subjects with high levels of protein in their urine (proteinuria).

In some embodiments, described herein is a method of treating a fungal infection in a subject with Compound 1 comprising administering Compound 1, wherein the subject also has a kidney disease and wherein the administration of compound 1 delays, slows down or avoids the kidney from progressing to end-stage renal disease (ESRD).

Chronic kidney disease (CKD) refers to all five stages of kidney damage, from very mild damage in stage 1 to complete kidney failure in stage 5. The stages of kidney disease are based on how well the kidneys can filter waste and extra fluid out of the blood. In the early stages of kidney disease, your kidneys are still able to filter out waste from your blood. In the later stages, your kidneys must work harder to get rid of waste and may stop working altogether.

The way doctors measure how well kidneys filter waste from the blood is by the estimated glomerular filtration rate, or eGFR. The eGFR is a number based on a blood test for creatinine, a waste product in the blood.

The stages of kidney disease are based on the eGFR number.

Stage 1 CKD: eGFR 90 or Greater. Stage 1 CKD means mild kidney damage and an eGFR of 90 or greater.

Stage 2 CKD: eGFR Between 60 and 89. Stage 2 CKD means mild kidney damage and an eGFR between 60 and 89.

Stage 3 CKD: eGFR Between 30 and 59. Stage 3 CKD means an eGFR between 30 and 59. An eGFR between 30 and 59 means that there is some damage to the kidneys and the kidneys are not working as well as they should. Stage 3 is separated into two stages: Stage 3a means an eGFR between 45 and 59; Stage 3b means an eGFR between 30 and 44. Many people with Stage 3 kidney disease do not have any symptoms.

Stage 4 CKD: eGFR Between 15 and 29. Stage 4 CKD means an eGFR between 15 and 29. An eGFR between 15 and 30 means the kidneys are moderately or severely damaged and are not working as they should. Stage 4 kidney disease should be taken very seriously—it is the last stage before kidney failure.

Stage 5 CKD: eGFR Less than 15. Stage 5 CKD means an eGFR less than 15. An eGFR less than 15 means the kidneys are getting very close to failure or have completely failed. If the kidneys fail, waste builds up in the blood, which makes the afflicted person very sick.

In some embodiments, are methods for treating fungal infections in subjects with impaired kidney function. In some embodiments, the methods of treating comprise treatment regimens comprising the administration of Compound 1, or a pharmaceutically acceptable salt, solvate, or hydrate thereof, to a subject with a fungal infection. In some embodiments, a dose adjustment of the compound 1, or a pharmaceutically acceptable salt, solvate, or hydrate thereof that is administered to the subject is not required based on the kidney status of the subject. In some embodiments, the formulations that are administered are cyclodextrin free (i.e. do not include one or more cyclodextrin excipients).

### Fungal Diseases

In some embodiments, the fungal disease is selected from the group consisting of aspergillosis, blastomycosis, candidiasis, coccidioidomycosis (Valley Fever), cryptococcosis, histoplasmosis, mucormycosis, Pneumocystis pneumonia (PCP), ringworm, sporotrichosis, and talaromycosis.

In some embodiments, the fungal disease is aspergillosis. In some embodiments, aspergillosis is allergic bronchopulmonary aspergillosis (abpa), allergic aspergillus sinusitis, chronic pulmonary aspergillosis, invasive aspergillosis or cutaneous (skin) aspergillosis. In some embodiments, the subject has an aspergilloma.

In some embodiments, the fungal disease is blastomycosis.

In some embodiments, the fungal disease is candidiasis. In some embodiments, candidiasis is oropharyngeal candidiasis (thrush), vulvovaginal candidiasis (vaginal candidiasis), fungemia, or invasive candidiasis.

In some embodiments, the fungal disease is coccidioidomycosis (Valley Fever). In some embodiments, coccidioidomycosis is acute coccidioidomycosis (primary pulmonary coccidioidomycosis), chronic coccidioidomycosis, or disseminated coccidioidomycosis, including primary cutaneous coccidioidomycosis.

In some embodiments, the fungal disease is cryptococcosis. In some embodiments, cryptococcosis is wound or cutaneous cryptococcosis, pulmonary cryptococcosis, or cryptococcal meningitis. In some embodiments, the fungal disease is a fungal eye infection. In some embodiments, the fungal eye infection is fungal keratitis, fungal exogenous endophthalmitis, or fungal endogenous endophthalmitis.

In some embodiments, the fungal disease is histoplasmosis. In some embodiments, histoplasmosis is acute histoplasmosis. In some embodiments, histoplasmosis is chronic histoplasmosis.

In some embodiments, the fungal disease is mucormycosis. In some embodiments, mucormycosis is rhinocerebral (sinus and brain) mucormycosis, pulmonary (lung) mucormycosis, gastrointestinal mucormycosis, cutaneous (skin) mucormycosis, or disseminated mucormycosis.

In some embodiments, the fungal disease is Pneumocystis pneumonia (PCP).

In some embodiments, the fungal disease is ringworm. In some embodiments, the ringworm is tinea pedis, tinea cruris, tinea capitis, tinea barbae, tinea manuum, tinea unguium, or tinum corporis. In some embodiments, the ringworm is caused by a type of fungi including Trichophyton, Microsporum, or Epidermophyton.

In some embodiments, the fungal disease is sporotrichosis. In some embodiments, sporotrichosis is cutaneous (skin) sporotrichosis, pulmonary (lung) sporotrichosis, or disseminated sporotrichosis.

In some embodiments, the fungal disease is talaromycosis.

In some embodiments, the fungal disease or infection is caused by a Cryptococcus, Aspergillus, Candida, Coccidioides, Blastomyces, Ajellomyces, Histoplasma, Rhizopus, Apophysomyces, Absidia, Saksenaea, Rhizomucor pusillus, Entomophthora, Conidiobolus, Basidiobolus, Sporothrix, Pneumocystis jirovecii, Talaromyces marneffei, Asclepias, Fusarium, or Scedosporium fungus/species. In some embodiments, the fungal disease is caused by a fungal species including, but not limited to, Aspergillus fumigatus, Aspergillus flavus, Aspergillus niger, Aspergillus terreus, Blastomyces dermatitidis, Ajellomyces dermatitidis, Candida albicans, Candida auris, Candida glabrata, Candida parapsilosis, Candida rugosa, Candida tropicalis, Coccidioides immitis, Coccidioides posadasii, Cryptococcus neoformans, Cryptococcus gattii, Histoplasma capsulatum, Rhizopus stolonifer, Rhizopus arrhizus, Mucor indicus, Cunninghamella bertholletiae, Apophysomyces elegans, Absidia species, Saksenaea species, Rhizomucor pusillus, Entomophthora species, Conidiobolus species, Basidiobolus species, Sporothrix schenckii, Pneumocystis jirovecii, Talaromyces marneffei, Asclepias albicans, Fusarium solani, Scedosporium apiospermum, and Rhizomucor pusillus. In some embodiments, the fungal disease is caused by the fungal species Aspergillus fumigatus. In some embodiments, the fungal disease is caused by the fungal species Candida albicans. In some embodiments, the fungal disease is caused by the fungal species Fusarium solani. In some embodiments, the fungal disease is caused by the fungal species Mucor indicus. In some embodiments, the fungal disease is caused by the fungal species Scedosporium apiospermum. In some embodiments, the fungal disease is caused by the fungal species Cryptococcus neoformans. In some embodiments, the fungal disease is caused by the fungal species Cryptococcus gattii. In some embodiments, the fungal disease is caused by the fungal species Candida auris.

In some embodiments, the fungal disease or infection is caused by a Aspergillus fumigatus, Blastomyces, Ajellomyces, Candida, Coccidioides, Cryptococcus, Histoplasm, Rhizopus Muco, Cunninghamell, Apophysomyces, Absidi, Saksenaea, Entomophthora, Conidiobolus, Basidiobolus, Sporothrix, Pneumocystis, Talaromyces, Asclepias, Fusarium, Scedosporium fungus or from a fungus from the Mucorales order, or any combination thereof.

In some embodiments, the fungal disease or infection is caused by a Cryptococcus, Aspergillus, Candida, Fusarium, Scedosporium fungus or from a fungus from the Mucorales order, or any combination thereof. In some embodiments, the fungal disease or infection is caused by Aspergillus fumigatus, Aspergillus flavus, Blastomyces dermatitidis, Ajellomyces dermatitidi, Candida albican, Candida glabrata, Candida rugosa, Candida auris, Coccidioides immitis, Coccidioides posadasii, Cryptococcus neoformans, Cryptococcus gattii, Histoplasma capsulatum, Rhizopus stolornfer, Rhizopus arrhizus, Mucor indicus, Cunninghamella bertholletiae, Apophysomyces elegans, Absidia species, Saksenaea species, Rhizomucor pusillus, Entomophthora species, Conidiobolus species, Basidiobolus species, Sporothrix schenckii, Pneumocystis jirovecii, Talaromyces marneffei, Asclepias albicans, Fusarium solani, Scedosporium apiospermum, Rhizomucor pusillus, or any combination thereof.

In some embodiments, a compound described herein is active against the fungal Gwt1 protein. This conserved enzyme catalyzes the glycosylphosphatidyl inositol (GPI) post-translational modification that anchors eukaryotic cell surface proteins to the cell membrane. In yeasts, GPI mediates cross-linking of cell wall mannoproteins to β-1,6-glucan. Inhibition of this enzyme in both Candida albicans and Saccharomyces cerevisiae has been shown to result in inhibition of maturation and localization of GPI-anchored mannoproteins thus demonstrating pleiotropic effects that include inhibition of fungal adherence to surfaces, inhibition of biofilm formation, inhibition of germ tube formation, severe growth defects, or lethality.

### Subjects

In some embodiments, the subject is a human. In some embodiments, the subject is immunocompromised. In some embodiments, the subject is undergoing therapy with at least one immunosuppressant drug. In some embodiments, the immunosuppressant drug increases the risk of opportunistic infections in the subject.

Immunosuppressant agents/drugs that can weaken the immune system include, but are not limited to, corticosteroids, methotrexate, cyclosporine, tacrolimus, sirolimus, everolimus, pomalidomide, omalizumab, azathioprine, lenalidomide, thalidomide, anti-TNF inhibitors, interleukin inhibitors, Janus kinase inhibitors, Sphingosine-1-phosphate-receptor (S1P) agonists, S1P antagonists Calcineurin inhibitors, mTOR inhibitors, nucleotide synthesis inhibitors, biologics, and monoclonal antibodies.

Corticosteroids include, but are not limited to, prednisone, budesonide, prednisolone, methylprednisolone.

Janus kinase inhibitors include, but are not limited to, tofacitinib, baricitinib, filgotinib, and upadacitinib.

Sphingosine-1-phosphate-receptor antagonists include, but are not limited to, FTY720.

S1P agonists include, but are not limited to, ozanimod, etrasimod.

Calcineurin inhibitors include, but are not limited to, cyclosporine, and tacrolimus.

mTOR inhibitors include, but are not limited to, sirolimus, and everolimus.

Interleukin inhibitors include, without limitation, rilonacept, canakinumab, anakinra, reslizumab, brodalumab, ustekinumab, benralizumab, mepolizumab, tocilizumab, ixekizumab, dupilumab, secukinumab, tildrakizumab, guselkumab, sarilumab, basiliximab, risankizumab, siltuximab, daclizumab, and daclizumab.

Nucleotide synthesis inhibitors include, but are not limited to, azathioprine, leflunomide, mycophenolate.

Biologics include, but are not limited to, TNF alpha inhibitors, an integrin inhibitors, IL-12/23 inhibitors. Biologics include, but are not limited to, abatacept, adalimumab, anakinra, certolizumab, etanercept, golimumab, infliximab, ixekizumab, natalizumab, rituximab, secukinumab, tocilizumab, ustekinumab, etrolizumab, vedolizumab.

Monoclonal antibodies include, but are not limited to, basiliximab, daclizumab, alemtuzumab, rituximab, belatacept.

In some embodiments, the human subject is under the age of 1 year. In some embodiments, the human subject is an infant under 1 month old. In some embodiments, the human subject is over the age of 70 years. In some embodiments, the subject is infected with HIV/AIDS. In some embodiments, the subject is undergoing or has undergone cancer chemotherapy treatment. In some embodiments, the subject is undergoing or has undergone corticosteroid treatment. In some embodiments, the subject is undergoing or has undergone TNF inhibitor treatment. In some embodiments, the subject is a transplant recipient. In some embodiments, the subject is a recipient of a hematopoietic stem-cell transplant, bone marrow transplant, lung transplant, liver transplant, heart transplant, kidney transplant, pancreas transplant or a combination thereof. In some embodiments, the subject is a recipient of a hematopoietic stem-cell transplant. In some embodiments, the subject is a recipient of a bone marrow transplant. In some embodiments, the subject is a recipient of a lung transplant. In some embodiments, the subject is a recipient of a liver transplant. In some embodiments, the subject is a recipient of a heart transplant. In some embodiments, the subject is a recipient of a kidney transplant. In some embodiments, the subject is a recipient of a pancreas transplant.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “pharmaceutically acceptable salt” in reference to a compound refers to a salt of the compound, which does not cause significant irritation to a mammal to which it is administered and does not substantially abrogate the biological activity and properties of the compound. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. In some embodiments, pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability, in some cases, is manipulated as one aspect of delayed and sustained release behaviors. Also, because the salt-forming molecule, in some cases, is in equilibrium with a neutral form, passage through biological membranes, in some cases, is adjusted.

In some embodiments, pharmaceutically acceptable salts are generally prepared by reacting the free base with a suitable organic or inorganic acid or by reacting the acid with a suitable organic or inorganic base. The term may be used in reference to any compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. In some embodiments, when an acidic substituent is present, such as —CO₂H, ammonium, morpholinium, sodium, potassium, barium, or calcium salts, and the like are formed. In some embodiments, when a basic group is present, such as amino, or a basic heteroaryl ring, such as pyridyl, an acidic addition salt is formed, such as hydrochloride salt, hydrobromide salt, phosphate salt, sulfate salt, trifluoroacetate salt, trichloroacetate salt, acetate salt, oxalate salt, maleate salt, pyruvate salt, malonate salt, succinate salt, citrate salt, tartrate salt, fumarate salt, mandelate salt, benzoate salt, cinnamate salt, methanesulfonate salt, ethanesulfonate salt, picrate salt, and the like. Additional pharmaceutically acceptable salt forms of therapeutic agents are listed in Berge, et al., Journal of Pharmaceutical Sciences, Vol. 66(1), pp. 1-19 (1977).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The terms “administer,” “administering”, “administration,” and the like, as used herein, refer to the methods that in some cases enable delivery of compounds or compositions to the desired site of biological action. These methods include, but are not limited to oral routes, intraduodenal routes, and parenteral routes (including intravenous, intraperitoneal, intravascular, or infusion). Those of skill in the art are familiar with administration techniques that can be employed with the compounds and methods described herein. In some embodiments, the compounds and compositions described herein are administered orally. In some embodiments, the compounds and compositions described herein are administered intravenously. In some embodiments, the compounds and compositions described herein are administered by intravenous infusion.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

As used herein, the terms “fungal infection” or “fungal disease” refer to a disease caused by pathogenic fungi. The fungal infection may be opportunistic or a primary infection and may be caused by fungi that are yeasts and/or molds.

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound described herein, or a pharmaceutically acceptable salt, solvate, or hydrate thereof, and a co-agent, are both administered to a patient simultaneously in the form of a single entity or dosage. The term “non-fixed combination” means that the active ingredients, e.g., a compound described herein, or a pharmaceutically acceptable salt, solvate, or hydrate thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The terms “kit” and “article of manufacture” are used as synonyms.

The term “subject” or “patient” encompasses mammals. Examples of mammals include, but are not limited to, any member of the mammalian class: humans, non-human primates such as chimpanzees, and other apes and monkey species. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein the term “about” means within ±10% of the value.

### Methods of Use

In one embodiment, compound 1, or a pharmaceutically acceptable salt, solvate, or hydrate thereof, is used in the preparation of medicaments for the treatment of diseases or conditions caused by fungal infections in a mammal. Methods for treating any of the diseases or conditions described herein in a mammal in need of such treatment, involves administration of pharmaceutical compositions that include compound 1, or a pharmaceutically acceptable salt, solvate, or hydrate thereof, or active metabolite of compound 1 (i.e., compound 1A), in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing compound 1, or a pharmaceutically acceptable salt, solvate, or hydrate thereof, are administered to a patient susceptible to or otherwise at risk of a particular disease or condition. Such an amount is defined to be a “prophylactically effective amount or dose.” In this use, the precise amounts also depend on the patient's state of health, weight, and the like. When used in patients, effective amounts for this use will depend on the underlying risk of developing a fungal infection, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising compound 1, or a pharmaceutically acceptable salt, solvate, or hydrate thereof, in order to prevent a return of the symptoms of the disease or condition.

In certain embodiments wherein the patient's condition does not improve, upon the doctor's discretion the administration of compound 1, or a pharmaceutically acceptable salt, solvate, or hydrate thereof, is administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease or condition is retained. In certain embodiments, however, the patient requires intermittent treatment on a long-term basis upon any recurrence of symptoms.

In one aspect, compound 1, or a pharmaceutically acceptable salt, solvate, or hydrate thereof, is administered daily to humans in need of therapy with compound 1, or a pharmaceutically acceptable salt, solvate, or hydrate thereof. In some embodiments, compound 1, or a pharmaceutically acceptable salt, solvate, or hydrate thereof, is administered once a day. In some embodiments, compound 1, or a pharmaceutically acceptable salt, solvate, or hydrate thereof, is administered twice a day.

In some embodiments, compound 1, or a pharmaceutically acceptable salt, solvate, or hydrate thereof, is administered twice daily, e.g., morning and evening.

In some embodiments, compound 1, or a pharmaceutically acceptable salt, solvate, or hydrate thereof, is administered for at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 1 month, at least 2 months, at least 3 months, at least 4 months, or more.

In some embodiments, compound 1, or a pharmaceutically acceptable salt, solvate, or hydrate thereof, is administered to the human on a continuous dosing schedule. In some embodiments, compound 1, or a pharmaceutically acceptable salt, solvate, or hydrate thereof, is administered to the human on a continuous daily dosing schedule.

The term “continuous dosing schedule” refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, daily for a week followed by a week of no administration of the therapeutic agent, daily for two weeks followed by one or two weeks of no administration of the therapeutic agent, daily for three weeks followed by one, two, or three weeks of no administration of the therapeutic agent, daily for four weeks followed by one, two, three, or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day.

The term “continuous daily dosing schedule” refers to the administration of a particular therapeutic agent every day at roughly the same time each day. In some embodiments, daily administration is once a day. In some embodiments, daily administration is twice a day. In some embodiments, daily administration is three times a day. In some embodiments, daily administration is more than three times a day.

In some embodiments, the amount of compound 1, or a pharmaceutically acceptable salt, solvate, or hydrate thereof, is administered once a day. In some other embodiments, the amount of compound 1, or a pharmaceutically acceptable salt, solvate, or hydrate thereof, is administered twice a day.

In certain embodiments wherein improvement in the status of the disease or condition in the human is not observed, the daily dose of compound 1, or a pharmaceutically acceptable salt, solvate, or hydrate thereof, is increased. In some embodiments, a once a day dosing schedule is changed to a twice a day dosing schedule. In some embodiments, the frequency of administration is increased in order to provide maintained or more regular exposure to compound 1, or a pharmaceutically acceptable salt, solvate, or hydrate thereof. In some embodiments, the frequency of administration is increased in order to provide repeat high Cₘₐₓ levels on a more regular basis and provide maintained or more regular exposure to compound 1, or a pharmaceutically acceptable salt, solvate, or hydrate thereof, or active metabolite of compound 1 (i.e., compound 1A), such as a higher AUC level. In some embodiments, the frequency of administration is increased in order to provide maintained or more regular exposure to compound 1A. In some embodiments, the frequency of administration is increased in order to provide repeat high Cₘₐₓ levels on a more regular basis and provide maintained or more regular exposure to compound 1A, such as a higher AUC level.

In any of the aforementioned aspects are further embodiments comprising single administrations of the effective amount of compound 1, or a pharmaceutically acceptable salt, solvate, or hydrate thereof, including further embodiments in which compound 1, or a pharmaceutically acceptable salt, solvate, or hydrate thereof, is administered (i) once a day; or (ii) multiple times over the span of one day.

In any of the aforementioned aspects are further embodiments comprising multiple administrations of the effective amount of compound 1, or a pharmaceutically acceptable salt, solvate, or hydrate thereof, including further embodiments in which (i) compound 1 is administered continuously or intermittently: as in a single dose; (ii) the time between multiple administrations is every 6 hours; (iii) compound 1 is administered to the mammal every 8 hours; (iv) compound 1 is administered to the mammal every 12 hours; (v) compound 1 is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

Generally, a suitable dose of compound 1, or a pharmaceutically acceptable salt, solvate, or hydrate thereof, for administration to a human will be in the range of about 500 mg/day to about 2000 mg/day; from about 600 mg/day to about 2000 mg/day; from about 800 mg/day to about 2000 mg/day; or from about 1000 mg/day to about 2000 mg/day.

In some embodiments, the administrations of the effective amount of compound 1, or a pharmaceutically acceptable salt, solvate, or hydrate thereof, comprises a treatment regimen that comprises the administration of a loading dose of compound 1, or a pharmaceutically acceptable salt, solvate, or hydrate thereof, followed by a maintenance dose of compound 1, or a pharmaceutically acceptable salt, solvate, or hydrate thereof. In some embodiments, the loading dose of compound 1, or a pharmaceutically acceptable salt, solvate, or hydrate thereof, is administered in a different manner than the maintenance dose of compound 1, or a pharmaceutically acceptable salt, solvate, or hydrate thereof. In some embodiments, the loading dose of compound 1, or a pharmaceutically acceptable salt, solvate, or hydrate thereof, is administered in the same manner than the maintenance dose of compound 1, or a pharmaceutically acceptable salt, solvate, or hydrate thereof.

In some embodiments, the loading dose is administered as a solution by intravenous (I.V.) infusion.

In some embodiments, the maintenance doses are administered orally in the form of solid dosage forms. In some embodiments, the solid dosage forms are tablets. In some embodiments, the maintenance doses are administered as a solution by intravenous (I.V.) infusion.

In some embodiments, the loading dose comprises about 1500 mg to about 2500 mg of compound 1, or a pharmaceutically acceptable salt, solvate, or hydrate thereof. In some embodiments, the loading dose comprises about 2000 mg of compound 1, or a pharmaceutically acceptable salt, solvate, or hydrate thereof. In some embodiments, the loading dose of compound 1, or a pharmaceutically acceptable salt, solvate, or hydrate thereof, comprises the administration of two doses of compound 1, or a pharmaceutically acceptable salt, solvate, or hydrate thereof, to the subject by intravenous (I.V.) infusion. In some embodiments, each loading dose of compound 1, or a pharmaceutically acceptable salt, solvate, or hydrate thereof, is administered to the subject by intravenous (I.V.) infusion over about 30 minutes to about 3 hours. In some embodiments, each loading dose of compound 1, or a pharmaceutically acceptable salt, solvate, or hydrate thereof, is administered to the subject by intravenous (I.V.) infusion over about 30 minutes, over about 45 minutes, over about 1 hour, over about 1.5 hours, over about 2 hours, over about 2.5 hours, over about 3 hours, or over more than 3 hours. In some embodiments, each of the two doses of the loading dose comprises about 1000 mg of compound 1, or a pharmaceutically acceptable salt, solvate, or hydrate thereof.

In some embodiments, the loading dose comprises administration of about 1000 mg of compound 1, or a pharmaceutically acceptable salt, solvate, or hydrate thereof, to the subject by intravenous (I.V.) infusion followed by a second administration of about 1000 mg of compound 1, or a pharmaceutically acceptable salt, solvate, or hydrate thereof, to the subject by intravenous (I.V.) infusion within about 24 hours of the first infusion. In some embodiments, the second loading dose is administered within about 12 hours of the first loading dose, within about 13 hours of the first loading dose, within about 14 hours of the first loading dose, within about 15 hours of the first loading dose, within about 16 hours of the first loading dose, within about 17 hours of the first loading dose, within about 18 hours of the first loading dose, within about 19 hours of the first loading dose, within about 20 hours of the first loading dose, within about 21 hours of the first loading dose, within about 22 hours of the first loading dose, within about 23 hours of the first loading dose, or within about 24 hours of the first loading dose.

In some embodiments, the maintenance doses are initiated on the second day of treatment. In some embodiments, the maintenance dose is administered once daily starting on the second day of treatment.

In some embodiments, each maintenance dose comprises once daily administration of about 1000 mg to about 2000 mg of compound 1, or a pharmaceutically acceptable salt, solvate, or hydrate thereof. In some embodiments, each maintenance dose comprises once daily administration of about 600 mg to about 1000 mg of compound 1, or a pharmaceutically acceptable salt, solvate, or hydrate thereof. In some embodiments, each maintenance dose comprises once daily administration of about 600 mg of compound 1, or a pharmaceutically acceptable salt, solvate, or hydrate thereof. In some embodiments, each maintenance dose comprises once daily administration of about 650 mg of compound 1, or a pharmaceutically acceptable salt, solvate, or hydrate thereof. In some embodiments, each maintenance dose comprises once daily administration of about 700 mg of compound 1, or a pharmaceutically acceptable salt, solvate, or hydrate thereof. In some embodiments, each maintenance dose comprises once daily administration of about 750 mg of compound 1, or a pharmaceutically acceptable salt, solvate, or hydrate thereof. In some embodiments, each maintenance dose comprises once daily administration of about 800 mg of compound 1, or a pharmaceutically acceptable salt, solvate, or hydrate thereof. In some embodiments, each maintenance dose comprises once daily administration of about 850 mg of compound 1, or a pharmaceutically acceptable salt, solvate, or hydrate thereof. In some embodiments, each maintenance dose comprises once daily administration of about 900 mg of compound 1, or a pharmaceutically acceptable salt, solvate, or hydrate thereof. In some embodiments, each maintenance dose comprises once daily administration of about 950 mg of compound 1, or a pharmaceutically acceptable salt, solvate, or hydrate thereof. In some embodiments, each maintenance dose comprises once daily administration of about 1000 mg of compound 1, or a pharmaceutically acceptable salt, solvate, or hydrate thereof. In some embodiments, each maintenance dose comprises once daily administration of about 1050 mg of compound 1, or a pharmaceutically acceptable salt, solvate, or hydrate thereof. In some embodiments, each maintenance dose comprises once daily administration of about 1100 mg of compound 1, or a pharmaceutically acceptable salt, solvate, or hydrate thereof. In some embodiments, each maintenance dose comprises once daily administration of about 1150 mg of compound 1, or a pharmaceutically acceptable salt, solvate, or hydrate thereof. In some embodiments, each maintenance dose comprises once daily administration of about 1200 mg of compound 1, or a pharmaceutically acceptable salt, solvate, or hydrate thereof. In some embodiments, each maintenance dose comprises once daily administration of more than about 1200 mg of compound 1, or a pharmaceutically acceptable salt, solvate, or hydrate thereof.

In some embodiments, each maintenance dose of compound 1, or a pharmaceutically acceptable salt, solvate, or hydrate thereof is administered over a period of about 30 minutes to about 3 hours by I.V. infusion starting on the second, third, or fourth day of treatment. In some embodiments, each maintenance dose of compound 1, or a pharmaceutically acceptable salt, solvate, or hydrate thereof is administered over a period of about 30 minutes, over about 45 minutes, over about 1 hour, over about 1.5 hours, over about 2 hours, over about 2.5 hours, over about 3 hours, or over more than 3 hours by I.V. infusion starting on the second, third, or fourth day of treatment.

In some embodiments, maintenance doses of about 600 mg to about 1500 mg compound 1, or a pharmaceutically acceptable salt, solvate, or hydrate thereof are administered over a period of about 30 minutes to about 5 hours by I.V. infusion starting on the second, third, or fourth day of treatment. In some embodiments, maintenance doses of about 600 mg, about 650 mg, about 700 mg, about 750 mg, about 800 mg, about 850 mg, about 900 mg, about 950 mg, about 1000 mg, about 1050 mg, about 1100 mg, about 1150 mg, or about 1200 mg compound 1, or a pharmaceutically acceptable salt, solvate, or hydrate thereof are administered over a period of about 30 minutes to about 5 hours by I.V. infusion starting on the second, third, or fourth day of treatment.

In some embodiments, maintenance doses of about 600 mg to about 900 mg compound 1, or a pharmaceutically acceptable salt, solvate, or hydrate thereof are administered over a period of about 30 minutes to about 3 hours by I.V. infusion starting on the second, third, or fourth day of treatment. In some embodiments, maintenance doses of about 600 mg, about 650 mg, about 700 mg, about 750 mg, about 800 mg, about 850 mg, or about 900 mg compound 1, or a pharmaceutically acceptable salt, solvate, or hydrate thereof are administered over a period of about 30 minutes to about 3 hours by I.V. infusion starting on the second, third, or fourth day of treatment.

In some embodiments, each maintenance dose comprises once daily administration of more than about 900 mg of compound 1, or a pharmaceutically acceptable salt, solvate, or hydrate thereof. In some embodiments, each maintenance dose comprises once daily administration of about 900 mg to about 2000 mg of compound 1, or a pharmaceutically acceptable salt, solvate, or hydrate thereof. In some embodiments, maintenance doses of about 900 mg to about 2000 mg compound 1, or a pharmaceutically acceptable salt, solvate, or hydrate thereof are administered over a period of about 30 minutes to about 3 hours by I.V. infusion starting on the second, third, or fourth day of treatment. In some embodiments, maintenance doses of about 900 mg to about 2000 mg compound 1, or a pharmaceutically acceptable salt, solvate, or hydrate thereof are administered over a period of more than 3 hours by I.V. infusion starting on the second, third, or fourth day of treatment.

In some embodiments, the maintenance dose of compound 1, or a pharmaceutically acceptable salt, solvate, or hydrate thereof is administered orally to the subject starting on the second, third, or fourth day of treatment.

In some embodiments, the maintenance dose of about 800 mg to about 1000 mg of compound 1, or a pharmaceutically acceptable salt, solvate, or hydrate thereof is administered orally once daily to the subject starting on the second, third, or fourth day of treatment. In some embodiments, the maintenance dose of about 800 mg of compound 1, or a pharmaceutically acceptable salt, solvate, or hydrate thereof is administered orally once daily to the subject starting on the second, third, or fourth day of treatment. In some embodiments, the maintenance dose of about 900 mg of compound 1, or a pharmaceutically acceptable salt, solvate, or hydrate thereof is administered orally once daily to the subject starting on the second, third, or fourth day of treatment. In some embodiments, the maintenance dose of about 1000 mg of compound 1, or a pharmaceutically acceptable salt, solvate, or hydrate thereof is administered orally once daily to the subject starting on the second, third, or fourth day of treatment.

In some embodiments, starting on the second, third, or fourth day of treatment: a) about 600 mg to about 900 mg of compound 1, or a pharmaceutically acceptable salt, solvate, or hydrate thereof is administered over a period of about 30 minutes to about 3 hours by I.V. infusion; or b) about 800 mg to about 1000 mg of compound 1, or a pharmaceutically acceptable salt, solvate, or hydrate thereof is administered orally once daily.

In some embodiments, starting on the second day of treatment, about 600 mg to about 900 mg of compound 1, or a pharmaceutically acceptable salt, solvate, or hydrate thereof is administered over a period of about 30 minutes to about 3 hours by I.V. infusion; and starting on the fourth day of treatment: a) about 600 mg to about 900 mg of compound 1, or a pharmaceutically acceptable salt, solvate, or hydrate thereof is administered over a period of about 30 minutes to about 3 hours by I.V. infusion; or b) about 800 mg to about 1000 mg of compound 1, or a pharmaceutically acceptable salt, solvate, or hydrate thereof is administered orally once daily.

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the individual subject, the severity of the disease or condition being treated, the identity (e.g., weight) of the human, and the particular additional therapeutic agents that are administered (if applicable), and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of compound 1 lies within a range of circulating concentrations that include the ED₅₀ with minimal toxicity. In certain embodiments, the daily dosage range and/or the unit dosage amount varies within this range depending upon the dosage form employed and the route of administration utilized.

In some embodiments, methods for treating fungal infections in a mammal with compound 1, or a pharmaceutically acceptable salt, solvate, or hydrate thereof, results in improvements of clinical symptoms attributed to the infection, improvements in radiologic abnormalities, and resolution of fungemia, if present. In some embodiments, clinical symptoms attributed to the infection include, for example, general appearance including appearance of the skin, head, eyes, ears, nose, throat, neck, trunk, or lymph nodes, or the respiratory, cardiovascular, gastrointestinal, genitourinary, musculoskeletal, neurological, psychological, lymphatic/hematological, and endocrine/metabolic systems of the mammal.

In some embodiments, improvements in one or more outcome measures are by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the administration of compound 1, or a pharmaceutically acceptable salt, solvate, or hydrate thereof, to a mammal with a fungal infection or a mold infection results in one or more outcome measures improving by at least or about 0.5 fold, 1 fold, 1.5 fold, 2 fold, 2.5 fold, 3 fold, 3.5 fold, 4 fold, 5 fold, 6 fold, 7 fold, 8 fold, 9 fold, 10 fold, or more than 10 fold. Improvements, in some embodiments, are compared to a control. In some embodiments, a control is an individual who does not receive compound 1, or a pharmaceutically acceptable salt, solvate, or hydrate thereof. In some embodiments, the control is an individual who does not receive a full dose of compound 1, or a pharmaceutically acceptable salt, solvate, or hydrate thereof. In some embodiments, the control is baseline for the individual prior to receiving compound 1, or a pharmaceutically acceptable salt, solvate, or hydrate thereof.

In some embodiments, methods for treating a fungal infection or mold infection in a subject with compound 1, or a pharmaceutically acceptable salt, solvate, or hydrate thereof, results in improvements in one or more outcome measures. In some embodiments, a baseline assessment is determined, typically prior to the administration of compound 1, or a pharmaceutically acceptable salt, solvate, or hydrate thereof. Improvements in outcome measures are assessed with repeated assessments taken during treatment with compound 1 and a comparison against the baseline assessment and/or any prior assessment(s).

Evaluating patients for fungal infections and assessing efficacy of treatment with compound 1, or a pharmaceutically acceptable salt, solvate, or hydrate thereof, includes multiple modalities of diagnostic testing, including: radiological assessments including CT scanning of the chest, sinuses, and abdomen; fungal culture and microscopy of respiratory specimens; blood, serum, or bronchoalveolar fluid fungal antigen testing; blood, serum, or bronchoalveolar fluid pathogenic DNA testing; biopsy of the lung (open, percutaneous or transbronchial); the aspergillosis urine test; and other molecular testing of respiratory samples.

In some embodiments, methods for treating fungal infections in a mammal with compound 1, or a pharmaceutically acceptable salt, solvate, or hydrate thereof, result in improvements that are measured with a radiological assessment such as a computed tomography (CT) scan. In some embodiments, imaging methods that detect inflammation are used, such as positron emission tomography or indium-labeled white blood cell scintigraphy. In some embodiments, the radiological assessment is used to determine if a fungal infection is present.

In some embodiments, the radiological assessment is used to determine the size or extent of the infection. In some embodiments, CT scans are preformed every 7 days or 14 days while the mammal is undergoing treatment with compound 1, or a pharmaceutically acceptable salt, solvate, or hydrate thereof. In some embodiments, total infection load decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% following a treatment regimen with compound 1, or a pharmaceutically acceptable salt, solvate, or hydrate thereof.

In some embodiments, methods for treating fungal infections in a mammal with compound 1, or a pharmaceutically acceptable salt, solvate, or hydrate thereof, result in improvements that are measured with a fungal culture of a bodily fluid, such as bronchoalveolar fluid, sputum, bronchial brush, or sinus aspirate. In some embodiments, fungal load as determined in a fungal culture decreases by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% following a treatment regimen with compound 1, or a pharmaceutically acceptable salt, solvate, or hydrate thereof.

In some embodiments, methods for treating fungal infections in a mammal with compound 1, or a pharmaceutically acceptable salt, solvate, or hydrate thereof, result in improvements that are measured with a suitable pathogen DNA test. In some embodiments, pathogen DNA levels are measured in blood samples from the mammal. In some embodiments, pathogen DNA levels are measured in serum samples from the mammal. In some embodiments, pathogen DNA levels are measured in bronchoalveolar lavage fluid samples from the mammal. In some embodiments, pathogen DNA levels are determined using a known pathogen DNA detection assay. In some embodiments, pathogen DNA levels are determined using next-generation sequencing and/or polymerase chain reaction (PCR) analysis. In some embodiments, pathogen DNA levels decrease by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% following a treatment regimen with compound 1, or a pharmaceutically acceptable salt, solvate, or hydrate thereof.

In some embodiments, methods for treating fungal infections in a subject with compound 1, or a pharmaceutically acceptable salt, solvate, or hydrate thereof, results in histological improvements in biopsied tissue samples taken from a subject with a fungal infection or mold infection. In some embodiments, the biopsied tissue is lunge tissue.

In some embodiments, methods for treating fungal infections in a mammal with compound 1, or a pharmaceutically acceptable salt, solvate, or hydrate thereof, increases the overall survival rate of the subject by at least 10%, at least 20%, at least 30%, at least 40%, at least 50%, at least 60%, at least 70%, at least 80%, at least 90%, at least 95%, or 100%. In some embodiments, the overall survival rate is measured after 42 days. In some embodiments, the overall survival rate is measured after 84 days.

In some embodiments, methods for treating fungal infections in a mammal with compound 1, or a pharmaceutically acceptable salt, solvate, or hydrate thereof, decreases the all-cause mortality rate of the subject by at least 10%, at least 20%, at least 30%, at least 40%, at least 50%, at least 60%, at least 70%, at least 80%, at least 90%, at least 95%, or 100%. In some embodiments, the all-cause mortality rate is measured after 42 days. In some embodiments, the all-cause mortality rate is measured after 84 days.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into formulations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered in combination with pharmaceutically acceptable carriers, excipients or diluents, in a pharmaceutical composition. Administration of the compounds and compositions described herein is carried out by any method that enables delivery of the compounds to the site of action. These methods include, though are not limited to delivery via enteral routes (including oral, gastric or duodenal feeding tube) or parenteral routes (injection or infusion), although the most suitable route, in some cases, depends upon, for example, the condition and disease of the recipient.

In some embodiments, compound 1 or a pharmaceutically acceptable salt, solvate, or hydrate thereof, is included within a pharmaceutical composition. As used herein, the term “pharmaceutical composition” refers to a liquid or solid composition that contains a pharmaceutically active ingredient (e.g., compound 1 or a pharmaceutically acceptable salt, solvate, or hydrate thereof) and at least a carrier, where none of the ingredients is generally biologically undesirable at the administered quantities.

Pharmaceutical compositions incorporating compound 1, or a pharmaceutically acceptable salt, solvate, or hydrate thereof, take any physical form that is pharmaceutically acceptable. In some embodiments, pharmaceutical compositions described herein are in a suitable form for oral administration. In one embodiment of such pharmaceutical compositions, a therapeutically effective amount of compound 1, or a pharmaceutically acceptable salt, solvate, or hydrate thereof, is incorporated.

In some embodiments, conventional inert ingredients and manner of formulating the pharmaceutical compositions are used. In some embodiments, known methods of formulating the pharmaceutical compositions are followed. All of the usual types of compositions are contemplated, including, but not limited to, tablets, capsules, and solutions. The amount of compound 1, or a pharmaceutically acceptable salt, solvate, or hydrate thereof, however, is best defined as the effective amount, that is, the amount of compound 1, or a pharmaceutically acceptable salt, solvate, or hydrate thereof, that provides the desired dose to the subject in need of such treatment.

In some cases, capsules are prepared by mixing compound 1, or a pharmaceutically acceptable salt, solvate, or hydrate thereof, with a suitable diluent and filling the proper amount of the mixture in capsules. The usual diluents include inert powdered substances such as starch of many different kinds, powdered cellulose, especially crystalline, and microcrystalline cellulose, sugars such as fructose, mannitol, and sucrose, grain flours, and similar edible powders.

In some cases, tablets are prepared by direct compression, by wet granulation, or by dry granulation. Their formulations usually incorporate diluents, binders, lubricants, and disintegrators, as well as compound 1, or a pharmaceutically acceptable salt, solvate, or hydrate thereof. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin, and sugars such as lactose, fructose, glucose, and the like. Natural and synthetic gums are also convenient, including acacia, alginates, methylcellulose, polyvinylpyrrolidine, and the like. In some cases, polyethylene glycol, ethylcellulose, and waxes serve as binders.

In some cases, a lubricant in a tablet formulation helps prevent the tablet and punches from sticking in the die. In some cases, a lubricant is chosen from such solids as talc, magnesium and calcium stearate, stearic acid, and hydrogenated vegetable oils.

Tablet disintegrators are substances that swell when wetted to break up the tablet and release the compound. They include starches, clays, celluloses, aligns, and gums. More particularly, tablet disintegrators include corn and potato starches, methylcellulose, agar, bentonite, wood cellulose, powdered natural sponge, cation-exchange resins, alginic acid, guar gum, citrus pulp, carboxymethylcellulose, and sodium lauryl sulfate.

Enteric formulations are often used to protect an active ingredient from the strongly acidic contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. Tablets can also be coated to provide a desired color.

In some embodiments, pharmaceutical compositions for use in any of the methods provided herein are described in the Examples.

### Combination Treatments

In certain instances, it is appropriate to administer compound 1, or a pharmaceutically acceptable salt, solvate, or hydrate thereof, in combination with one or more other therapeutic agents.

In one embodiment, the therapeutic effectiveness of compound 1, or a pharmaceutically acceptable salt, is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering compound 1, or a pharmaceutically acceptable salt, solvate, or hydrate thereof, with another agent (which also includes a therapeutic regimen) that also has therapeutic benefit.

In one specific embodiment, compound 1, or a pharmaceutically acceptable salt, solvate, or hydrate thereof, is co-administered with a second therapeutic agent, wherein compound 1, or a pharmaceutically acceptable salt, and the second therapeutic agent modulate different aspects of the disease or condition being treated, thereby providing a greater overall benefit than administration of either therapeutic agent alone.

In any case, regardless of the disease or condition being treated, the overall benefit experienced by the patient is simply additive of the two therapeutic agents or the patient experiences a synergistic benefit.

In certain embodiments, different therapeutically-effective dosages of compound 1, or a pharmaceutically acceptable salt, solvate, or hydrate thereof, will be utilized in formulating pharmaceutical composition and/or in treatment regimens when compound 1, or a pharmaceutically acceptable salt, solvate, or hydrate thereof, is administered in combination with one or more additional agent, such as an additional therapeutically effective drug, an adjuvant or the like. Therapeutically effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of compound 1, or a pharmaceutically acceptable salt or solvate thereof, is initiated prior to, during, or after treatment with a second agent described herein, and continues until any time during treatment with the second agent or after termination of treatment with the second agent. It also includes treatments in which compound 1, or a pharmaceutically acceptable salt, solvate, or hydrate thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g., the disease or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, compound 1, or a pharmaceutically acceptable salt or solvate thereof, is administered either simultaneously with the one or more other therapeutic agents, or sequentially.

In combination therapies, the multiple therapeutic agents (one of which is compound 1, or a pharmaceutically acceptable salt, solvate, or hydrate thereof) are administered in any order or even simultaneously. If administration is simultaneous, the multiple therapeutic agents are, by way of example only, provided in a single, unified form, or in multiple forms (e.g., as a single pill or as two separate pills; or as a single IV infusion solution or as two separate IV infusion solutions).

Compound 1, or a pharmaceutically acceptable salt, solvate, or hydrate thereof, as well as combination therapies, are administered before, during or after the occurrence of a disease or condition, and the timing of administering the composition containing compound 1, or a pharmaceutically acceptable salt, solvate, or hydrate thereof, varies. Thus, in one embodiment, compound 1 or a pharmaceutically acceptable salt, solvate, or hydrate thereof, is used as a prophylactic and are administered continuously to subjects with a propensity to develop conditions or diseases in order to prevent the occurrence of the disease or condition. In another embodiment, compound 1, or a pharmaceutically acceptable salt, solvate, or hydrate thereof, is administered to a subject during or as soon as possible after the onset of the symptoms. In specific embodiments, compound 1, or a pharmaceutically acceptable salt or solvate thereof, is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, compound 1, or a pharmaceutically acceptable salt or solvate thereof, or a formulation containing compound 1, or a pharmaceutically acceptable salt or solvate thereof, is administered for at least 4 weeks, at least 6 weeks, at least 8 weeks, at least 10 weeks, at least 12 weeks, or more than 12 weeks.

### Exemplary Agents for Use in Combination Therapy

In some embodiments, compound 1, or a pharmaceutically acceptable salt, solvate, or hydrate thereof, is administered in combination with one or more additional therapies used for treating fungal and/or mold infections in a mammal.

In certain embodiments, the at least one additional therapy is administered at the same time as compound 1, or a pharmaceutically acceptable salt, solvate, or hydrate thereof. In certain embodiments, the at least one additional therapy is administered less frequently than compound 1, or a pharmaceutically acceptable salt, solvate, or hydrate thereof. In certain embodiments, the at least one additional therapy is administered more frequently than compound 1, or a pharmaceutically acceptable salt, solvate, or hydrate thereof. In certain embodiments, the at least one additional therapy is administered prior to administration of compound 1, or a pharmaceutically acceptable salt, solvate, or hydrate thereof. In certain embodiments, the at least one additional therapy is administered after administration of compound 1, or a pharmaceutically acceptable salt, solvate, or hydrate thereof.

In some embodiments, the at least one additional therapy is an antifungal agent. In some embodiments, the second therapeutic agent is an antifungal agent selected from the group consisting of: a polyene antifungal agent, an azole antifungal agent, an allylamine antifungal agent, and an echinocandin antifungal agent.

In some embodiments, the polyene antifungal agent is amphotericin B, candicidin, filipin, hamycin, natamycin, nystatin, or rimocidin.

In some embodiments, the azole antifungal agent is an imidazole, a triazole, or a thiazole. In some embodiments, the imidazole is bifonazole, butoconazole, clotrimazole, econazole, fenticonazole, ketoconazole, luliconazole, miconazole, omoconazole, oxiconazole, sertaconazole, sulconazole, or tioconazole. In some embodiments, the triazole is albaconazole, efinaconazole, epoxiconazole, fluconazole, isavuconazole, itraconazole, posaconazole, propiconazole, ravuconazole, terconazole, or voriconazole. In some embodiments, the thiazole is abafungin.

In some embodiments, the allylamine antifungal agent is amorolfin, butenafine, naftifine, or terbinafine.

In some embodiments, the echinocandin antifungal agent is selected from the group consisting of: anidulafungin, caspofungin, micafungin and rezafungin.

### Adjunctive Therapies

In addition to antifungal treatment, the optimal management of patients with fungal infections includes surgical debulking of infected tissues and removal of venous catheters in the occasional patient with confirmed catheter-related fungal infections. In some embodiments, treatment with compound 1, or a pharmaceutically acceptable salt, solvate, or hydrate thereof, comprises G-CSF or GM-CSF, G-CSF-stimulated granulocyte transfusions. In some embodiments, treatment with compound 1, or a pharmaceutically acceptable salt, solvate, or hydrate thereof, comprises gamma interferon.

### Kits and Articles of Manufacture

Described herein are kits for treating treatment of a fungal infection in a subject comprising administering to said subject compound 1, or a pharmaceutically acceptable salt, solvate, or hydrate thereof.

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. In some embodiments, the containers are formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatment regimens that would benefit from the administration of compound 1, or a pharmaceutically acceptable salt, solvate, or hydrate thereof.

The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In some embodiments, a label is on or associated with the container. A label, in some cases, is on a container when letters, numbers or other characters forming the label are attached, molded or etched into the container itself; a label, in some cases, is associated with a container when it is present within a receptacle or carrier that also holds the container, e.g., as a package insert. A label, in some cases, is used to indicate that the contents are to be used for a specific therapeutic application. The label, in some cases, indicates directions for use of the contents, such as in the methods described herein.

In certain embodiments, a pharmaceutical composition comprising compound 1, or a pharmaceutically acceptable salt, solvate, or hydrate thereof, is presented in a pack or dispenser device which, in some cases, contains one or more unit dosage forms. The pack, in some cases, for example contains metal or plastic foil, such as a blister pack. The pack or dispenser device, in some cases, is accompanied by instructions for administration. The pack or dispenser, in some cases, is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, in some cases, is the labeling approved by the U.S. Food and Drug Administration for prescription drugs, or the approved product insert. Compositions containing a compound provided herein formulated in a compatible pharmaceutical carrier, in some cases, is also prepared, placed in an appropriate container, and labeled for treatment of an indicated condition.

### EXAMPLES

The following examples are provided for illustrative purposes only and not to limit the scope of the claims provided herein.

### Example 1: Compound 1 Injection

Compound 1 Injection is prepared as a sterile solution that is further diluted into 0.9% sodium chloride injection prior to administration. Compound 1 Injection is a solution formulated at a concentration of 20 mg/mL. The formulation consists of compound 1 drug substance, sodium chloride, potassium phosphate (dibasic and monobasic), hydrochloric acid, sodium hydroxide, and Water for Injection (WFI).

A 50-mL sterile glass vial is filled with 35 mL of Compound 1 Injection yielding 700 mg/vial. Compound 1 Injection is further diluted and administered as an IV infusion as specified in the clinical protocol. During the preparation of the admixture solution containing compound 1, Compound 1 Injection is filtered with a 0.2 μm filter prior to infusion to remove any inherent particles.

Table 1 describes the composition of Compound 1 Injection, 20 mg/mL, for a 35 mL fill in a 50 mL vial.

TABLE 1

| | Approximate | | | |
|---|---|---|---|---|
| Component | Concentration | | Content Per Unit | |
| Compound 1 | 20 | mg/mL | 700 | mg |
| Sodium Chloride | 6.1 | mg/mL | 213.5 | mg |
| Potassium Phosphate, Monobasic | 0.16 | mg/mL | 5.6 | mg |
| Potassium Phosphate, Dibasic | 3.27 | mg/mL | 114.5 | mg |

| | | |
|---|---|---|
| Sodium Hydroxide | As needed | As needed |
| Hydrochloric Acid | As needed | As needed |
| Water for Injection | N/A | Q.S. 35 mL |

N/A = not applicable;
QS = quantity sufficient for

To make the Compound 1 Injection Formulation: 1. Add sodium chloride, potassium phosphate (monobasic and dibasic) into a vessel containing water for injection; 2. Adjust pH to 8.0 using 1M hydrochloric acid solutions and/or 1M sodium hydroxide solutions; 3. Slowly add compound 1 drug substance to the solution and stir/mix at 15° C. to 30° C.; 4. Adjust pH to 8.0 using hydrochloric acid solutions and/or sodium hydroxide solutions to dissolve; 5. Q.S. with water for injection and continue to stir at 15° C. to 30° C.; 6. Aseptically filter through 2×0.2 m membrane filters into sterile 50 mL vials with a 35 mL fill volume closed with a chlorobutyl stopper. (Note: At the completion of each manufactured batch, the filter integrity is tested using a bubble point test and result is documented in the batch record); and 7. The vials are inspected prior to packaging and labeling.

### Example 2: Compound 1 Tablets

Compound 1 Tablets are formulated at strengths of 100 mg and 200 mg coated white tablets. Table 2 and Table 3 list the content of the Compound 1 Tablets at 100 mg and 200 mg strength, respectively.

TABLE 2

| Component | Amount per Tablet (% wt) | | Function |
|---|---|---|---|
| Compound 1 | 100 mg | (25.0%) | Active |
| | | | Ingredient |
| Avicel DG³ | 231 mg | (57.75%¹) | Diluent |
| Pregelatinized Starch | 40 mg | (5.0%¹, 5%²) | Disintegrant |
| Colloidal silicon dioxide | 3 mg | (0.75%²) | Glidant |
| (Cab-o-sil M5P) | | | |
| Povidone | 12 mg | (3.0%¹) | Disintegrant |
| Talc | 8 mg | (2.0%¹) | Glidant |
| Magnesium Stearate | 6 mg | (0.5%¹, 1.00%²) | Lubricant |
| (HyQual) Vegetable Source | | | |
| Core Weight (mg) | 400 mg | | |
| Opadry II AMB, coating | 20 mg | (5.0%) | Film Coating |
| white⁴ | | | |

| | | |
|---|---|---|
| Purified Water | Not applicable | Solvent |
| Coated Tablet Weight (mg) | 420 mg | |

¹intragranular.
²extragranular.
³Avicel DG is comprised or 75% of microcrystalline cellulose and 25% anhydrous dibasic calcium phosphate.
⁴The film coating Opadry II AMB is manufactured by ColorCon and composed of polyvinyl alcohol, talc, titanium dioxide, glyceryl monocaprylocaprate, and sodium lauryl sulfate.

TABLE 3

| Component | Amount per Tablet (% wt) | | Function |
|---|---|---|---|
| Compound 1 | 200 mg | (25.0%) | Active |
| | | | Ingredient |
| Avicel DG³ | 462 mg | (57.75%¹) | Diluent |
| Pregelatinized Starch | 80 mg | (5.0%¹, 5%²) | Disintegrant |
| Colloidal silicon dioxide | 6 mg | (0.75%²) | Glidant |
| (Cab-o-sil M5P) | | | |
| Povidone | 24 mg | (3.0%¹) | Disintegrant |
| Talc | 16 mg | (2.0%¹) | Glidant |
| Magnesium Stearate | 12 mg | (0.5%¹, 1.00%²) | Lubricant |
| (HyQual) Vegetable Source | | | |
| Core Weight (mg) | 800 mg | | |
| Opadry II AMB, coating | 40 mg | (5.0%) | Film Coating |
| white⁴ | | | |

| | | |
|---|---|---|
| Purified Water | Not applicable | Solvent |
| Coated Tablet Weight (mg) | 840 mg | |

¹intragranular.
²extragranular.
³ Avicel DG is comprised or 75% of microcrystalline cellulose and 25% anhydrous dibasic calcium phosphate.
⁴The film coating Opadry II AMB is manufactured by ColorCon and composed of polyvinyl alcohol, talc, titanium dioxide, glyceryl monocaprylocaprate, and sodium lauryl sulfate.

### Example 3: An Open-Label Study to Evaluate the Efficacy and Safety of Compound 1 in Non-Neutropenic Patients with Candidemia, with or without Invasive Candidiasis, Inclusive of Patients with Suspected Resistance to Standard of Care Antifungal Treatment

The need for improved treatment of IFDs remains high, particularly with the growing number of immunocompromised patients, such as hematopoietic stem cell and solid organ transplant recipients, who are at particular risk for developing these infections and in whom treatment can be complex. Species of Candida and Aspergillus are recognized as two major causes of fungal diseases in these patients, although other emerging fungi, such as non-C. Albicans (e.g., C. glabrata and C. auris), Fusarium spp., Scedosporium spp., and Mucorales fungi, are contributing to the need to find new and better strategies for managing these infections. Existing antifungal agents can be difficult to use, are often poorly tolerated, or have become increasingly ineffective due to the rise of drug resistant fungal strains.

Administration of Compound 1 in a concentration of 1000 mg IV BID will be administered on Day 1, 600 mg IV QD will be administered on Days 2 through 3, and then subsequently 600 mg IV or 700 mg PO will be administered throughout the Study Drug Treatment Period. This treatment may have advantages over standard of care (SOC) therapy against certain resistant fungal diseases, where SOC treatment might show no or limited therapeutic effectiveness.

### Primary Objective

The primary objective of this study is to evaluate the efficacy and safety of Compound 1 for the treatment of adult non-neutropenic patients 18 to 80 years of age (inclusive) with candidemia that may include patients with suspected or confirmed resistance to SOC antifungal treatment.

### Secondary Objectives

The secondary objectives of this study are to:
Evaluate the time to first negative blood culture
Evaluate the percentage of patients with Mycological Outcomes at the End of Study Drug Treatment (EOST), End of Antifungal Treatment (EOT), and 2 and 4 weeks after EOT
Evaluate the percentage of patients with Treatment Success at EOT, and 2 and 4 weeks after EOT
Evaluate overall survival at Study Day 30
Evaluate safety parameters, including number of patients with TEAEs
Evaluate PK parameters of Compound 1

### Summary of Study Design

This is a multicenter, open-label, non-comparative, single-arm study to evaluate the efficacy and safety of Compound 1 for the first-line treatment for candidemia including suspected or confirmed antifungal-resistant candidemia in non-neutropenic patients 18 to 80 years of age (inclusive). Suspicion of antifungal-resistant candidemia is sufficient and subsequent documented resistance is not required for enrollment. The Study Drug Treatment Period will be up to a maximum of 14 days (inclusive of the loading dose [Study Day 1]). After completion of 14 days study drug therapy, if further antifungal treatment is indicated to complete treatment of candidemia in accordance with standard practice guidelines, fluconazole (unless susceptibility results warrant alternative antifungal therapy) may commence for up to a further 7 days. There will be a Follow-up Period of 4 weeks (+4 days) after EOT. The total duration of participation in the study is up to approximately 7.5 weeks (inclusive of the Screening Period [≤96 hours prior to Baseline]).

Patients with a yeast identified in a blood culture or a positive rapid diagnostic method are eligible to be consented and screened for the study. Patients must have at least 1 positive blood test for Candida spp. (or yeast suspected to be Candida) for a diagnosis of candidemia to be considered for enrollment into the study. Patients with a positive blood culture showing yeast suspected to be Candida must have identification of Candida spp. from positive blood culture confirmed prior to dosing. Screening and Baseline procedures and the start of Compound 1 study drug will be initiated within 96 hours from the time that the SOC blood sample for the Candida spp. positive culture or rapid diagnostic test was drawn. Patients with 2 days (>48 hours) equivalent of prior systemic antifungal treatment at approved doses to treat the current episode of candidemia within 96 hours before first dose will be excluded. However, patients with Candida infections proven to be resistant to the specific antifungal administered may have received ≤5 days (≤120 hours) equivalent of that prior treatment (results of susceptibility testing are required prior to enrollment).

Patients with >2 days (>48 hours) equivalent of prior systemic antifungal treatment at approved doses to treat the current episode of candidemia within 96 hours before first dose will be excluded. However, patients with Candida infections proven to be resistant to the specific antifungal administered may have received ≤5 days (≤120 hours) equivalent of that prior treatment (results of susceptibility testing are required prior to enrollment).

On Study Day 1 (or over the first 24 hours if started in the evening), a 1000 mg Compound 1 loading dose will be administered over 3 hours by IV infusion BID. On Study Days 2 and 3 of study drug, a 600 mg Compound 1 maintenance dose will be administered over 3 hours by IV infusion QD. On Study Day 4 and onward, the Compound 1 maintenance dose will be administered as either 600 mg Compound 1 IV infusion QD over 3 hours or 700 mg PO QD. Patients who have completed a minimum of 3 days of IV Compound 1, are clinically stable as determined by the Investigator, able to swallow tablets, and have no further growth of the infecting organism 48 hours following the most recent blood culture, may switch from IV to PO dosing on Study Day 4 and onward. Study drug will be administered for a maximum of 14 days. At the Investigator's discretion, patients requiring a longer duration of antifungal therapy will be switched to fluconazole (unless susceptibility results warrant alternative antifungal therapy), to adhere to the IDSA clinical practice guidelines for the treatment of Candidiasis. Candida spp. bloodstream infection will be monitored by daily blood culture during Study Drug Treatment until 2 consecutive blood cultures are negative, and at EOST, EOT, and 2 and 4 weeks after EOT, or Early Termination. Simultaneously drawn blood samples will be collected for Candida testing by T2 magnetic resonance (T2MR) assay at Baseline, during Study Drug Treatment, and EOST, or Early Termination. Other cultures, histopathology, and imaging tests to assess the site(s) and extent of candidemia infection at other sites will be conducted as clinically indicated, and the results should be recorded in the electronic Case Report Form (eCRF). The management of intravascular catheters, intravascular devices, and, if applicable, any drains will be recorded, including any associated microbiology results. Patients will be monitored for safety throughout the duration of the study.

Plasma samples for PK (Compound 1 [prodrug] and Compound 1A [active moiety]) will be collected at Baseline (pre dose), twice weekly during Study Drug Treatment, EOST, EOT, 2 weeks after EOT, or Early Termination. Serum samples for (1,3)-β-D-glucan levels will be collected at Baseline (pre-dose) and EOST, or Early Termination (if applicable).

The evaluation of treatment outcome will be assessed at EOST, EOT, and 2 and 4 weeks after EOT, or Early Termination. The end of study will occur after the last visit of the last patient on the study.

Indication: Treatment of non-neutropenic patients with candidemia, inclusive of those patients with suspected or confirmed antifungal-resistant candidemia

### Population:

This study will enroll male and female patients 18 to 80 years of age (inclusive) with a new diagnosis of candidemia (positive blood test for Candida spp.).

### Inclusion Criteria:

Patients must meet all of the following criteria to be eligible for study entry:
1. Male or female 18 to 80 years of age (inclusive)
2. New diagnosis of candidemia based on a blood sample drawn within 96 hours of dosing with:
   a. Positive blood culture for Candida spp., including those Candida spp. with suspected (in the opinion of the Investigator) or documented resistance to at least 1 SOC systemic antifungal agent; or
   b. Positive result from a Sponsor-approved rapid diagnostic blood test for Candida spp. infection (a rapid diagnostic test may be used to begin eligibility assessments; however, a subsequent confirmatory blood culture is required prior to dosing of Compound 1)
3. Able to have pre-existing intravascular catheters removed and replaced (if necessary)

### Dosing Criteria

Patients must meet the following criteria to begin dosing:
1. Confirmed diagnosis of candidemia
2. Received ≤2 days (≤48 hours) equivalent of prior systemic antifungal treatment at approved doses to treat the current episode of candidemia OR ≤5 days (≤120 hours) equivalent of prior treatment for candidemia caused by Candida spp. with documented resistance to the specific prior antifungal administered.

### Treatment Groups

All patients will be administered a 1000 mg Compound 1 loading dose BID followed by a 600 mg Compound 1 maintenance dose QD on Study Day 2 and Study Day 3. From Study Day 4 onwards, the Compound 1 maintenance dose will be administered as either 600 mg Compound 1 IV infusion over 3 hours QD or may be switched to 700 mg PO QD when/if the criteria for PO dosing are met

### Dose

In PK-PD studies, immunocompromised mice were infected with one of three spp. of Candida (C. albicans, C. glabrata, or C. auris) and groups of animals were dosed with Compound 1 at different dose fractionations. The AUC/MIC ratio was determined to be the PK-PD variable that best correlated with antifungal efficacy as assessed by fungal burden (colony-forming units [CFUs]) in the kidney. The probability of target attainment (PTA) was calculated separately for each Candida spp. tested. The PTA calculation used the Compound 1A free drug AUC level at the stasis endpoint divided by the MIC required to inhibit the growth of 90% of organisms (MIC₉₀) of each of the Candida spp. tested. The AUC level was estimated from a population PK model derived primarily from the Phase 1 PK data. The stasis endpoint was defined as the quantity of Candida spp. in CFUs just prior to Compound 1 administration compared to CFUs at the endpoint of assessment (i.e., 24 hours for C. albicans; 96 hours for C. glabrata and C. auris). The MIC data for the Candida strains tested were obtained from recent surveillance data. Using the AUC at the stasis endpoint, along with the MIC90 from the surveillance data and the predicted exposure at the dose regimen to be used in this study, the PTA for the three Candida spp. tested was shown to be approximately 100%. Further, sensitivity analyses were conducted to evaluate the PTA under different scenarios including increased variability of PK parameters and higher Candida spp. MIC₉₀ values. For both scenarios the PTA remained >90%.

In 2 Phase 1 studies in healthy volunteers, Compound 1 IV and PO formulations were safe and well tolerated. The majority of the TEAEs were mild, transitory, and resolved without intervention. No DLTs were observed. Specifically, in the FIH Phase 1 clinical study, a loading dose of Compound 1 1000 mg IV 2-hour infusion BID on Day 1, followed by a maintenance dose of Compound 1 600 mg IV 1-hour infusion QD on Days 2 through 7, was safe and well tolerated. This IV dose regimen is identical to the IV dose regimen that will be used in this study. In the second Phase 1 clinical study, a dose of Compound 1 at 1000 mg administered PO QD on Days 1 through 14 was safe and well tolerated. This PO-dose regimen is higher than the 700 mg PO dose that will be used in this study.

### Schedule

To ensure the safety and tolerability of Compound 1 dosing for 14 days, this study will use a Compound 1 dose and infusion duration already studied in Phase 1 for 14 days of therapy inclusive of IV and PO investigational drug therapy. The loading dose 1000 mg IV BID over a 3-hour infusion followed by 600 mg IV QD over a 3-hour infusion will optimize patient safety and tolerability on study. At Study Day 4, provided a patient meets the protocol-defined criteria for a PO switch, then the switch will occur to PO Compound 1 dose at 700 mg QD for no more than 14 days of combined IV and PO Compound 1 therapy.

### Randomization and Blinding

This is a non-randomized, open-label study.

### Drug Supplies

Formulation and Packaging Compound 1 Injection is formulated at a concentration of 20 mg/mL. The formulation consists of Compound 1 drug substance, sodium chloride, potassium phosphate (dibasic and monobasic), hydrochloric acid, sodium hydroxide, and sterile water for injection. A 50 mL sterile vial is filled with 35 mL of Compound 1 Injection. Compound 1 Injection will be reconstituted and administered as an IV infusion. Preparation and dilution instructions will be provided in the Pharmacy Manual.

Compound 1 Tablets are formulated at strengths of 100 mg and 200 mg white-coated tablets. The formulation consists of Compound 1 drug substance, microcrystalline cellulose, anhydrous dibasic calcium phosphate, colloidal silicon dioxide, pregelatinized starch, povidone, talc, magnesium

### Study Drug Administration

On Study Day 1 (or over the first 24 hours if started in the evening), a 1000 mg Compound 1 loading dose will be administered over 3 hours by IV infusion BID. On Study Days 2 and 3 of study drug, a 600 mg Compound 1 maintenance dose will be administered over 3 hours by IV infusion QD. On Study Day 4 and onward, the Compound 1 maintenance dose will be administered as either 600 mg Compound 1 IV infusion QD over 3 hours or 700 mg PO QD. Patients who have completed a minimum of 3 days of IV Compound 1, are clinically stable as determined by the Investigator, able to swallow tablets, and have no further growth of the infecting organism 48 hours following the most recent blood culture, may switch from IV to PO dosing on Study Day 4 and onward. Study drug will be administered for a maximum of 14 days (inclusive of the loading dose [Study Day 1]). Tablets are to be administered at the same time each day, whole, and taken by mouth with water within 30 minutes of being removed from the refrigerator. No splitting or crushing of tablets is allowed. If antifungal treatment is indicated for longer than 14 days, fluconazole may commence at the Investigator's discretion (unless susceptibility results warrant alternative antifungal therapy) for up to a further 7 days of therapy, to adhere to IDSA clinical practice guidelines for the treatment of Candidiasis.

### At End of Antifungal Treatment (EOT):

After completion of 14 days study drug therapy, if further antifungal treatment is indicated to complete treatment of candidemia in accordance with standard practice guidelines, fluconazole (unless susceptibility results warrant alternative antifungal therapy) may commence for up to a further 7 days. If applicable, an assessment of efficacy will also be made at the end of this antifungal treatment at EOT. Treatment Success is defined as meeting all of the following criteria:
Two consecutive blood cultures negative for Candida spp.
Alive at EOT
No additional systemic antifungal therapies (except for protocol-allowed step-down treatment [e.g., fluconazole]) through EOT
Treatment Failure is defined as any case that does not meet the criteria for Treatment Success.

Mycological Outcome:

Eradication is defined as a negative blood culture(s) for Candida spp. in the absence of additional antifungal therapy (except for protocol-allowed step-down treatment [e.g., fluconazole]) through EOT

At Follow-Up (2 Weeks and 4 Weeks after End of Antifungal Treatment):

Recurrence (mycological) is defined as a mycologically confirmed infection based on blood culture with the same Baseline Candida spp. during the 4 weeks after EOT. Relapse (DRC Assessment) is defined as re-occurrence of Candida in blood culture during the Follow-up Period, or diagnostic parameters indicative of recurrence or late spread of the Candida infection

### Results

A total of 21 patients were enrolled in the study: 20 were included in the mITT. Median duration of Compound 1 therapy was 11 days (range 5 to 14). All patients received IV Compound 1, 48% (10/21) received PO Compound 1. The DRC-assessed success rate at EOST was 80% (16/20). Survival at day 30 was 85% (17/20); deaths were not related to Compound 1. Compound 1 was well-tolerated with no treatment-related serious adverse events or discontinuations. Compound 1 had potent in vitro activity against all Candida spp. from this study, including isolates resistant to other antifungal agents.

Results in Patients with Renal Insufficiency: Subset Analysis

14/21 (66%) subjects had some degree of renal insufficiency: 7 had mild renal insufficiency (GFR:60-89), 5 had moderate renal insufficiency (GFR:30-59), and 2 had severe renal insufficiency (GFR:15-29). Most (12/14) completed study treatment. Treatment success rate at EOST in patients with renal insufficiency was 86% (12/14). None had a worsening of renal function at EOST. 4 patients had worsening of renal function during the follow-up period. Renal impairment did not increase exposure of FMGX. There were no treatment-related adverse events.

The examples and embodiments described herein are for illustrative purposes only and various modifications or changes suggested to persons skilled in the art are to be included within the spirit and purview of this application and scope of the appended claims.

1. A method of treating a fungal infection in a subject, the method comprising administering to a subject with a fungal infection a therapeutically effective amount of compound 1: 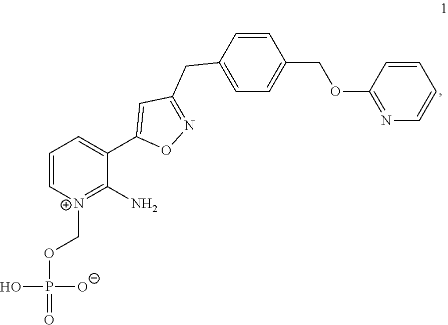 [SMILES: Nc1c(-c2cc(Cc3ccc(COc4ccccn4)cc3)no2)ccc[n+]1COP(=O)([O-])O]
or a pharmaceutically acceptable salt, solvate, or hydrate thereof;
wherein,
the fungal infection in the subject is caused by Candida spp., Aspergillus spp., Scedosporium spp., Fusarium spp., Paecilomyces spp., Purpureocillium spp., Dematiaceous spp., Rhizopus, Mucor spp., Lichtheimia spp., Cunninghamella spp., Acremonium spp., Rasamsonia spp., Scedosporium spp., Schizophyllum spp., Trichoderma spp., Alternaria spp., Cladophialophora spp., Cladosporium spp., Exophiala spp., Fonsecaea spp., Lomentospora spp., Phialophora spp., Scopulariopsis spp., Magnusiomyces (Geotrichum) spp., Trichosporon spp., Malassezia spp., Saprochaete spp., Kodamaea spp., Rhodotorula spp., Saccharomyces spp., Pseudozyma spp., Sporobolomyces spp., Exophiala spp., Lacazia spp., Emmonsia spp., Wickerhamomyces (Pichia) spp., Emergomyces spp., Talaromyces spp., or Emmonsia-like fungi, or a combination thereof, the therapeutically effective amount of compound 1 provides a steady state 24-hr Area Under the Concentration-Time Curve (AUC₀₋₂₄) in the subject of at least about 100 μg×hr/mL of compound 1A: 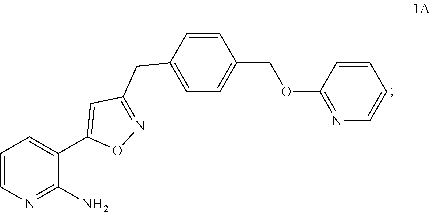 [SMILES: Nc1ncccc1-c1cc(Cc2ccc(COc3ccccn3)cc2)no1]
wherein the subject has a contradiction to standard of care antifungal therapy that is due to reduced kidney function or a kidney disease in the subject; and
wherein the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks.

2. (canceled)

3. (canceled)

4. The method of claim 1, wherein the kidney disease is chronic kidney disease (CKD), metabolic syndrome, vesicoureteral reflux, tubulointerstitial renal fibrosis, IgA nephropathy, diabetic nephropathy, Alport syndrome, HIV associated nephropathy, glomerular nephritis (GN), focal segmental glomerulosclerosis, membranous glomerulonephritis, mesangiocapillary GN, interstitial fibrosis and tubular atrophy (IFTA), acute kidney injury (AKI), acute obstructive nephropathy, or drug induced fibrosis.

5. The method of claim 4, wherein the kidney disease is chronic kidney disease (CKD).

6. (canceled)

7. The method of claim 1, wherein the subject has high levels of protein in his or her urine (proteinuria).

8. The method of claim 1, wherein the therapeutically effective amount of compound 1 provides a steady state 24-hr Area Under the Concentration-Time Curve (AUC₀₋₂₄) of at least 150 μg×hr/mL of the compound 1A.

9. (canceled)

10. The method of claim 1, wherein the contradiction to standard of care antifungal therapy comprises amphotericin B, candicidin, filipin, hamycin, natamycin, nystatin, rimocidin, bifonazole, butoconazole, clotrimazole, econazole, fenticonazole, isavuconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, abafungin, amorolfin, butenafine, naftifine, or terbinafine, anidulafungin, caspofungin, micafungin, rezafungin, or a pharmaceutically acceptable salt of any of the preceding antifungal agents.

11. The method of claim 10, wherein the fungal infection is caused by Candida spp., Aspergillus spp., Scedosporium spp., Fusarium spp., Paecilomyces spp., Purpureocillium spp., Dematiaceous spp., or Mucorales fungi, or a combination thereof.

12. The method of claim 10, wherein the subject is immunocompromised, is infected with HIV/AIDS, or has cancer.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. The method of claim 1, wherein the fungal infection is a cutaneous infection, lung infection, sinus infection, central nervous system infection, brain infection, eye infection, heart infection, kidney infection, gastrointestinal tract infection, stomach infection, pelvic infection, blood infection, or a combination thereof.

25. (canceled)

26. The method of claim 1, wherein the treatment regimen comprises a loading dose of compound 1, or a pharmaceutically acceptable salt, solvate, or hydrate thereof, and a maintenance dose of compound 1, or a pharmaceutically acceptable salt, solvate, or hydrate thereof.

27. The method of claim 26, wherein the treatment regimen comprises a loading dose of compound 1, or a pharmaceutically acceptable salt, solvate, or hydrate thereof, of about 2000 mg compound 1, or a pharmaceutically acceptable salt, solvate, or hydrate thereof.

28. The method of claim 27, wherein the loading dose of compound 1, or a pharmaceutically acceptable salt, solvate, or hydrate thereof, is administered to the subject by intravenous (I.V.) infusion.

29. The method of claim 27, wherein the loading dose of compound 1, or a pharmaceutically acceptable salt, solvate, or hydrate thereof, comprises the administration of two doses of compound 1, or a pharmaceutically acceptable salt, solvate, or hydrate thereof, to the subject by intravenous (I.V.) infusion.

30. The method of claim 27, wherein each loading dose of compound 1, or a pharmaceutically acceptable salt, solvate, or hydrate thereof, is administered to the subject by intravenous (I.V.) infusion over about 30 minutes to about 4 hours.

31. The method of claim 27, wherein each dose of the loading dose comprises about 1000 mg of compound 1, or a pharmaceutically acceptable salt, solvate, or hydrate thereof.

32. The method of claim 27, wherein the loading dose comprises administration of about 1000 mg of compound 1, or a pharmaceutically acceptable salt, solvate, or hydrate thereof, to the subject by intravenous (I.V.) infusion followed by a second administration of about 1000 mg of compound 1, or a pharmaceutically acceptable salt, solvate, or hydrate thereof, to the subject by intravenous (I.V.) infusion within about 24 hours of the first infusion.

33. The method of claim 27, wherein the maintenance dose is administered once daily starting on the second day of treatment.

34. The method of claim 33, wherein the maintenance dose comprises once daily administration of about 600 mg to about 1500 mg of compound 1, or a pharmaceutically acceptable salt, solvate, or hydrate thereof.

35. The method of claim 27, wherein the maintenance dose of compound 1, or a pharmaceutically acceptable salt, solvate, or hydrate thereof is administered over a period of about 30 minutes to about 4 hours by I.V. infusion starting on the second, third, or fourth day of treatment.

36. The method of claim 27, wherein the maintenance dose of about 600 mg to about 1200 mg compound 1, or a pharmaceutically acceptable salt, solvate, or hydrate thereof is administered over a period of about 30 minutes to about 4 hours by I.V. infusion starting on the second, third, or fourth day of treatment.

37. The method of claim 27, wherein the maintenance dose of compound 1, or a pharmaceutically acceptable salt, solvate, or hydrate thereof is administered orally to the subject starting on the second, third, or fourth day of treatment.

38. The method of claim 27, wherein the maintenance dose of about 800 mg to about 1000 mg compound 1, or a pharmaceutically acceptable salt, solvate, or hydrate thereof is administered orally once daily to the subject starting on the second, third, or fourth day of treatment.

39. The method of claim 27, wherein, starting on the second, third, or fourth day of treatment:
a) about 600 mg to about 900 mg of compound 1, or a pharmaceutically acceptable salt, solvate, or hydrate thereof is administered over a period of about 30 minutes to about 3 hours by I.V. infusion; or
b) about 700 mg to about 1000 mg of compound 1, or a pharmaceutically acceptable salt, solvate, or hydrate thereof is administered orally once daily.

40. The method of claim 27, wherein:
starting on the second day of treatment, about 600 mg to about 900 mg of compound 1, or a pharmaceutically acceptable salt, solvate, or hydrate thereof is administered over a period of about 30 minutes to about 3 hours by I.V. infusion; and
starting on the fourth day of treatment:
a) about 600 mg to about 900 mg of compound 1, or a pharmaceutically acceptable salt, solvate, or hydrate thereof is administered over a period of about 30 minutes to about 3 hours by I.V. infusion; or
b) about 700 mg to about 1000 mg of compound 1, or a pharmaceutically acceptable salt, solvate, or hydrate thereof is administered orally once daily.

41. (canceled)

42. The method of claim 1, wherein:
the treatment regimen comprises the daily administration of compound 1, or a pharmaceutically acceptable salt, solvate, or hydrate thereof, for about 4 weeks to about 6 weeks.

43. The method of claim 1, wherein:
the treatment regimen comprises the daily administration of compound 1, or a pharmaceutically acceptable salt, solvate, or hydrate thereof, for about 4 weeks to about 12 weeks.

44. The method of claim 1, wherein:
the treatment regimen comprises a loading dose of compound 1, or a pharmaceutically acceptable salt, solvate, or hydrate thereof, and maintenance doses of compound 1, or a pharmaceutically acceptable salt, solvate, or hydrate thereof;
wherein the loading dose of compound 1, or a pharmaceutically acceptable salt, solvate, or hydrate thereof, comprises the administration of two doses of compound 1, or a pharmaceutically acceptable salt, solvate, or hydrate thereof, to the subject by intravenous (I.V.) infusion on the first day of therapy, wherein each dose comprises about 1000 mg of compound 1, or a pharmaceutically acceptable salt, solvate, or hydrate thereof;
followed by maintenance doses comprising once daily administration of about 600 mg of compound 1, or a pharmaceutically acceptable salt, solvate, or hydrate thereof by intravenous (I.V.) infusion for at least two days, followed by either:
once daily administration of about 600 mg of compound 1, or a pharmaceutically acceptable salt, solvate, or hydrate thereof, by intravenous (I.V.) infusion; or
once daily oral administration of about 700 mg of compound 1, or a pharmaceutically acceptable salt, solvate, or hydrate thereof.

45. The method of claim 44, wherein the treatment regimen comprises up to 14 days of administration of compound 1, or a pharmaceutically acceptable salt, solvate, or hydrate thereof.

46. (canceled)

47. The method of claim 1, wherein:
the treatment regimen increases the chances of survival for the subject, decreases galactomannan levels in the subject, decreases β-d-glucan levels in the subject, or a combination thereof.

48. (canceled)

49. A method of treating a fungal infection in a subject, the method comprising administering to a subject with a fungal infection a therapeutically effective amount of compound 1: 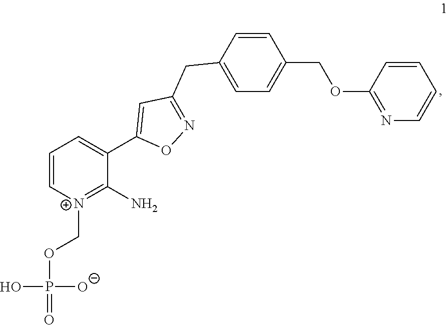 [SMILES: Nc1c(-c2cc(Cc3ccc(COc4ccccn4)cc3)no2)ccc[n+]1COP(=O)([O-])O]
or a pharmaceutically acceptable salt, solvate, or hydrate thereof;
wherein, the therapeutically effective amount of compound 1 provides a steady state 24-hr Area Under the Concentration-Time Curve (AUC₀₋₂₄) in the subject of at least about 100 μg×hr/mL of compound 1A: 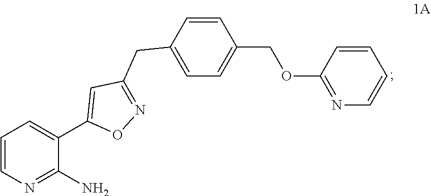 [SMILES: Nc1ncccc1-c1cc(Cc2ccc(COc3ccccn3)cc2)no1]
wherein the administration of compound 1, or a pharmaceutically acceptable salt, solvate, or hydrate thereof, to the subject comprises a treatment regimen comprising the daily administration of compound 1, or a pharmaceutically acceptable salt, solvate, or hydrate thereof, for at least 1-4 weeks;
wherein a dose adjustment of the compound 1, or a pharmaceutically acceptable salt, solvate, or hydrate thereof that is administered to the subject is not required based on the kidney status of the subject.
